SEQUENCE LISTING (37 sequences)

SEQ ID NO: 1 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - DNA, 25 bp, synthetic construct
  source 1..25: mol_type = other DNA
gatattgtgctcgacccagtctcca

SEQ ID NO: 3 - DNA, 21 bp, synthetic construct
  source 1..21: mol_type = other DNA
ggatacagttggtgcagcatc

SEQ ID NO: 4 - DNA, 18 bp, synthetic construct
  source 1..18: mol_type = other DNA
atgggatggagctggatc

SEQ ID NO: 5 - DNA, 27 bp, synthetic construct
  source 1..27: mol_type = other DNA
atagacagatgggggtgtcgttttggc

SEQ ID NO: 6 - protein, 25 aa, synthetic construct
QVTLKESGPGILQPSQTLSLTCSFS

SEQ ID NO: 7 - protein, 10 aa, synthetic construct
GFSLSSSGMG

SEQ ID NO: 8 - protein, 17 aa, synthetic construct
VGWIRQPSGKGLEWLTH

SEQ ID NO: 9 - protein, 7 aa, synthetic construct
IWWDDDK

SEQ ID NO: 10 - protein, 37 aa, synthetic construct
RYNPALKSRLTISKDTSNNQVFLKIASVDAADTATYY

SEQ ID NO: 11 - protein, 15 aa, synthetic construct
  VARIANT 8: note = X at position 8 is absent or is any naturally occurring amino acid residue
WSNNIWWXPYYFDYW

SEQ ID NO: 12 - protein, 10 aa, synthetic construct
GQGTTITVSS

SEQ ID NO: 13 - protein, 26 aa, synthetic construct
DIVLTQSPASLAVSLGQRATISYRAS

SEQ ID NO: 14 - protein, 10 aa, synthetic construct
KSVSTSGYSY

SEQ ID NO: 15 - protein, 17 aa, synthetic construct
MHWNQQKPGQPPRLLIY

SEQ ID NO: 16 - skipped

SEQ ID NO: 17 - protein, 35 aa, synthetic construct
NLESGVPARFSGSGSGTDFTLNIHPVEEEDAATYY

SEQ ID NO: 18 - protein, 10 aa, synthetic construct
  VARIANT 6: note = X at position 6 is absent or is any naturally occurring amino acid residue
CQHIRXAYTF

SEQ ID NO: 19 - protein, 9 aa, synthetic construct
GGGTKLEIK

SEQ ID NO: 20 - protein, 121 aa, synthetic construct
  VARIANT 104: note = X at position 104 is absent or is any naturally occurring amino acid residue
QVTLKESGPGILQPSQTLSLTCSFSGFSLSSSGMGVGWIRQPSGKGLEWLTHIWWDDDKRYNPALKSRLTISKDTSNNQVFLKIASVDAADTATYYWSNNIWWXPYYFDYWGQGTTITVSS

SEQ ID NO: 21 - protein, 110 aa, synthetic construct
  VARIANT 97: note = X at position 97 is absent or is any naturally occurring amino acid residue
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRXAYTFGGGTKLEIK

SEQ ID NO: 22 - protein, 25 aa, synthetic construct
QAVVTQESALTTSPGETVTLTCRSS

SEQ ID NO: 23 - protein, 9 aa, synthetic construct
TGAVTTSNY

SEQ ID NO: 24 - protein, 17 aa, synthetic construct
ANWVQEKPDHLFTGLIG

SEQ ID NO: 25 - skipped

SEQ ID NO: 26 - protein, 35 aa, synthetic construct
NRAPGVPARFSGSLIGDKAALTITGAQTEDEAIYF

SEQ ID NO: 27 - protein, 11 aa, synthetic construct
CALWYSNHLVF

SEQ ID NO: 28 - protein, 9 aa, synthetic construct
GGGTKLTVL

SEQ ID NO: 29 - protein, 25 aa, synthetic construct
QVTLKESGPGILQPSQTLSLTCSFS

SEQ ID NO: 30 - protein, 10 aa, synthetic construct
GFSLSSSGMG

SEQ ID NO: 31 - protein, 17 aa, synthetic construct
VGWIRQPSGKGLEWLTH

SEQ ID NO: 32 - protein, 7 aa, synthetic construct
IWWDDDK

SEQ ID NO: 33 - protein, 37 aa, synthetic construct; identical to SEQ ID NO: 10

SEQ ID NO: 34 - protein, 15 aa, synthetic construct
CGRIIYGGLYYFDYW

SEQ ID NO: 35 - protein, 10 aa, synthetic construct
GQGTTITVSS

SEQ ID NO: 36 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVL

SEQ ID NO: 37 - protein, 121 aa, synthetic construct
QVTLKESGPGILQPSQTLSLTCSFSGFSLSSSGMGVGWIRQPSGKGLEWLTHIWWDDDKRYNPALKSRLTISKDTSNNQVFLKIASVDAADTATYYCGRIIYGGLYYFDYWGQGTTITVSS

(19) United States
(12) Patent Application Publication
(10) Pub. No.: US 2025/0042983 A1
(43) Pub. Date: Feb. 6, 2025

(54) ANTI-LCN-2 ANTIBODIES AS A TREATMENT FOR EYE DISORDERS

(71) Applicant: UNIVERSITY OF PITTSBURGH - OF THE COMMONWEALTH SYSTEM OF HIGHER EDUCATION, Pittsburgh, PA (US)

(72) Inventors: Debasish SINHA, Clarksville, MD (US); Urvi GUPTA, Wexford, PA (US); Sayan GHOSH, Pittsburgh, PA (US); Peng SHANG, Pittsburgh, PA (US); Stacey HOSE, Clear Spring, MD (US); Nadezda Anatolyevna STEPICHEVA, Jeannette, PA (US); Jose Alain SAHEL, Pittsburgh, PA (US)

(21) Appl. No.: 18/717,623

(22) PCT Filed: Dec. 9, 2022
(86) PCT No.: PCT/US2022/081234

§ 371 Date: Jun. 7, 2024

Related U.S. Application Data
(60) Provisional application No. 63/348,583, filed on Jun. 3, 2022.
(60) Provisional application No. 63/288,348, filed on Dec. 10, 2021.

(51) Int. Cl. C07K 16/18; A61K 39/00; A61P 27/02

(52) U.S. Cl. CPC C07K 16/18; A61P 27/02; A61K 2039/505; C07K 2317/565; C07K 2317/76

(57) ABSTRACT

Disclosed are monoclonal antibodies, and antigen binding portions thereof, which neutralize human Lipocalin 2 (LCN-2). Related nucleic acids, recombinant expression vectors, host cells, populations of cells, and pharmaceutical compositions are also disclosed. Related methods of reducing or preventing ferroptotic death of retinal pigmented epithelium (RPE) cells, methods of reducing or preventing one or both of lipid peroxidation and inflammasome activation in RPE cells, methods of reducing or preventing a decrease in one or both of scotopic a- and b-wave responses in a retina, methods of increasing one or both of scotopic a- and b-wave responses in a retina, methods of increasing one or both of autophagy and glutathione peroxidase activity in RPE cells, and methods of treating or preventing age-related macular degeneration (AMD) are also disclosed.

### CROSS REFERENCE TO RELATED APPLICATIONS

This patent application claims the benefit of co-pending U.S. Provisional Patent Application No. 63/288,348, filed Dec. 10, 2021, and co-pending U.S. Provisional Patent Application No. 63/348,583, filed Jun. 3, 2022, each of which is incorporated by reference herein in its entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 34,172 Byte XML file named “764906.xml” created on Dec. 1, 2022.

### BACKGROUND OF THE INVENTION

Age-related macular degeneration (AMD) is the world's leading cause of blindness among the elderly. It is projected that the number of people with AMD worldwide will be 196 million in 2020, increasing to 288 million in 2040. More than 15 million Americans are affected by AMD, and the costs of treatment are in excess of $350 billion.

The vast majority of patients suffer from atrophic (dry) AMD. However, due to the relentless progression of the disease, within 10 years they will develop advanced disease. The burden of dry AMD is increasing as the “baby boomers” age. Despite this growing population of afflicted individuals, no definitive treatment (other than the AREDS II formulation for intermediate AMD) or prevention for dry AMD is currently available. The dry type of AMD affects approximately 80-90% of individuals with the disease. Its cause is unknown, and it usually progresses more slowly than the wet type.

Due to the large number of patients with atrophic AMD, there is a desire for new treatments.

### BRIEF SUMMARY OF THE INVENTION

An aspect of the invention provides an isolated or purified monoclonal antibody, or an antigen binding portion thereof, which neutralizes human Lipocalin 2 (LCN-2).

Aspects of the invention further provide nucleic acids, recombinant expression vectors, host cells, populations of cells, and pharmaceutical compositions relating to the inventive antibodies and antigen binding portions thereof.

Methods of reducing or preventing ferroptotic death of retinal pigmented epithelium (RPE) cells in a subject in need thereof, methods of reducing or preventing one or both of lipid peroxidation and inflammasome activation in RPE cells in a subject in need thereof, methods of reducing or preventing a decrease in one or both of scotopic a- and b-wave responses in a retina of a subject in need thereof, methods of increasing one or both of scotopic a- and b-wave responses in a retina of a subject in need thereof, methods of increasing one or both of autophagy and glutathione peroxidase activity in RPE cells in a subject in need thereof, and methods of treating or preventing AMD in a subject in need thereof are further provided by aspects of the invention.

### BRIEF DESCRIPTION OF THE SEVERAL VIEWS OF THE DRAWINGS

FIG. 1A shows an image of a gel (top) and a graph (bottom) demonstrating elevated levels of homodimer to monomer ratio in the RPE cells from 12 month old Cryba1 cKO mice, with an AMD-like phenotype. All values are Mean±SD. Significant changes are indicated by **P<0.01. n=4.

FIGS. 1B-1C show an image of a gel (1B) and a graph (1C) demonstrating that the homodimer variant is upregulated in the spent medium from floxed RPE explants overexpressing LCN-2 and is reduced to the monomer form upon beta-marcaptoethanol treatment. All values are Mean±SD. Significant changes are indicated by **P<0.01. n=4.

FIG. 1D shows an image of a gel (top) and a graph (bottom) demonstrating an increase in the fold change of the homodimer to monomer ratio in the RPE cells from human AMD donors compared to age-matched controls. All values are Mean±SD.

FIGS. 1E-1F show images resulting from an SD-OCT analysis (1E) and spider plot (1F) showing changes in the IS/OS+RPE layer in NOD-SCID mice sub-retinally injected with the spent medium (SM) from LCN-2 overexpressing floxed RPE explants. Treatment with mLCN-2 antibody (Clone #6) could protect from the structural alterations. All values are Mean±SD. Significant changes are indicated by *P<0.05, **P<0.01. n=4.

FIGS. 1G-1I show graphs of the results of electroretinography (ERG) studies (1G) showing alterations in both scotopic a- (1H) and b- (1I) wave responses. These results suggest alterations in retinal function in the LCN-2 overexpressing SM injected NOD-SCID mice, which was rescued upon treatment with mLCN-2 antibody (Clone #6). All values are Mean±SD. Significant changes are indicated by *P<0.05, **P<0.01. n=4.

FIG. 1J shows an image of a gel (top) and a graph (bottom) showing the results of experiments in which SDS-PAGE was followed by Western blot analysis using LCN-2 mAb (Clone #6) showing elevated levels of the LCN-2 homodimer in RPE cells from 12-month-old Cryba1 cKO mice, compared to floxed controls, which is reduced to the monomer form upon beta-mercaptoethanol treatment. All values are Mean±SD. Significant changes is indicated by **P<0.01.

FIG. 2A shows an image of a gel (top) and a graph (bottom) showing the results of experiments in which floxed RPE explants were infected with Adenovirus-GFP (Ad-GFP) or Adenovirus-LCN-2-GFP (Ad-LCN-2-GFP) constructs followed by co-immunoprecipitation using GFP-trap magnetic beads and Western blotting for ATG4B. The results showed binding between LCN-2 and ATG4B. n=4. All values are Mean±SD. Significant change is indicated by **P<0.01.

FIG. 2B is a graph of the results of a Pearsons' co-efficient analysis of ARPE19 cells infected with Ad-LCN-2-GFP and then stained with ATG4B antibody. The results showed co-localization between the two proteins n=15.

FIGS. 2C-2D are line (2C) and dot plot (2D) graphs showing the results of a thermal shift assay. The results showed an increase in melting temperature (Tm) of recombinant human ATG4B, when it was incubated with human LCN-2 protein (grey line), compared to only ATG4B protein (black line). The results indicate a binding affinity between the two proteins. The melting temperature is reflective of the change in sypro orange fluorescence (arbitrary units) which was added (5 μM) to each reaction mixture. n=4. All values are Mean±SD. Significant change is indicated by *P<0.05.

FIGS. 2E-2F show an image of a gel (2E) and a graph (2F) showing the results of a cell free Atg4B activity assay in vitro. The assay used recombinant human LC3-GST protein, incubated with recombinant human ATG4B (0.5 mg/ml), in the presence or absence of human recombinant LCN-2 (1.0 mg/ml) for 15 minutes at 37° C., followed by Western blotting with anti-GST antibody. The results showed that LCN-2 could inhibit LC3 processing through the inhibition of Atg4b activity as evident from decreased GST cleavage. The blot was over-exposed to allow evaluation of the levels of cleaved GST in the different conditions. n=4. All values are Mean±SD. Significant change is indicated by **P<0.01.

FIG. 2G shows a line graph (top) and a bar graph (bottom) showing the results of an experiment assessing ATG4B lipidation activity by transfecting ARPE19 cells with GFP-LC3-RFP-ΔLC3 construct and then either untreated, treated with recombinant LCN-2 for 24 h as a positive control, or infecting with an Ad-LCN-2 construct for 48 h followed by flow cytometry. An increase in the GFP/RFP ratio, which indicates a decrease in lipidation, was evident in recombinant LCN-2 treated and Ad-LCN-2 infected cells relative to control. n=4. All values are Mean±SD, while the results from the flow cytometry experiment are represented as Median±SD. Significant changes is indicated by *P<0.05.

FIG. 3A shows electron microscopy images of RPE cells showing abnormal accumulation of undigested outer segments (arrow in upper right) and double membrane autophagosomes in aged (20 months old) Cryba1 cKO mice (arrow in lower left and lower right), relative to age-matched floxed controls (upper left). Cryba1^{fl/fl} is upper left. Cryba1 cKO is upper right, lower left, and lower right. Scale bar=500 nm. n=6.

FIGS. 3B-3C show an image of a gel (3B) and a graph (3C) showing the results of a Western blot analysis for LC3-I and LC3-II in floxed RPE explants treated with Ad-LCN-2 construct. The results showed decreased autophagy flux (Ratio of LC3-II levels in chloroquine; ChQ treated cells to untreated cells) compared to untreated controls. n=4. All values are Mean±SD. Significant change is indicated by **P<0.01.

FIG. 3D is a graph showing the results of an experiment in which ARP19 cells were infected with the Ad-LCN-2 construct or left untreated (control) for 48 h, followed by an overnight infection with an Adenovirus-GFP-RFP-LC3B construct to infect these cells to label the autophagosomes and autolysosomes. The number of puncta (autophagosomes) were significantly inhibited in Ad-LCN-2 infected cells when compared with controls. Chloroquine was used a positive control and showed highly increased puncta, suggesting accumulation of the autophagosomes due to the dysfunctional lysosomes. n=4. All values are Mean±SD. Significant change is indicated by **P<0.01.

FIG. 3E is a dot representation of scRNAseq analysis from the sub-retinal region of 15-month-old Cryba1 cKO mice showing differential expression of several autophagy genes. In particular, genes which are required for autophagosome processing like AMBRA1, ATG4C, ATG9B, ATG7 and LC3A&B are downregulated in the RPE cell cluster, compared to age-matched floxed controls. n=4.

FIG. 3F is a graph showing the results of qPCR analysis showing decreased gene expression of Atg7, Atg9 and LC3b in 10-month-old Cryba1 cKO mice, compared to controls. n=4. **P<0.01

FIG. 4A is a graph showing the iron levels in RPE cells from young and aged Cryba1 cKO mice. The graph shows an increase in both fed and starved (16 hour) conditions compared to age-matched floxed controls. n=4. All values are Mean±SD. Significant change is indicated by **P<0.01.

FIGS. 4B-4C show an image of a gel (4B) and a graph (4C) showing the results of a Western blot analysis and densitometry. The results showed increased levels of NLRP3, cGAS and STING in the RPE cells of aged (10 months old) Cryba1 cKO mice, compared to age-matched controls. n=3. All values are Mean±SD. Significant change is indicated by **P<0.01.

FIG. 4D is a graph showing the results of an experiment in which RPE explants from WT and Cryba1 KO mice were grown in the presence of iron-rich medium containing 250 mM ferric ammonium citrate (FAC) for 72 h and infected with Ad-LCN-2 construct to simulate iron rich and high LCN-2 conditions in vivo. Assessment of iron levels in these cells revealed an increase in iron levels in Cryba1 KO RPE cells treated with FAC or infected with the LCN-2 construct or WT cells treated with FAC+Ad-LCN-2 and chloroquine. These results indicate that appropriate lysosomal function is involved in maintaining iron homeostasis in RPE cells. n=4. All values are Mean±SD. Significant change is indicated by **P<0.01.

FIG. 4E is a graph showing ELISA levels of IL-1b in 10 month old Cryba1 cKO RPE cells. The results showed an increase in comparison with age-matched floxed cells. n=4. All values are Mean±SD. Significant change is indicated by **P<0.01.

FIGS. 4F-41 show an image of gels (4F) and graphs (4G-4I) showing the results of an experiment in which RPE explants from WT, Cryba1 KO along with Sting KO or Sting^{GT}, mutant (which are animal models that do not express STING globally) were cultured in presence of FAC+Ad-LCN-2 with or without chloroquine. The results showed an increase in NLRP3 (4G) and IL-1b (4I) in the Cryba1 KO RPE cells treated with FAC and Ad-LCN-2. Similar treatment along with chloroquine addition to block lysosomal function in Sting KO or Sting^{GT} mutant RPE cells did not show an increase in either NLRP3 (4G) and IL-1b (4I). 4H shows the relative expression of Sting in WT and Cryba1 KO. n=4.

FIGS. 4J-4K show an image of gels (4J) and a graph (4K) showing the results of a Western blot analysis showing that treatment deferoxamine (Def; 10 mM for 24 h), an iron chelator and STING inhibitor (H-151; 1 mM for 12 h) rescued the levels of NLRP3 inflammasome expression in Cryba1 KO RPE cells treated with FAC and Ad-LCN-2 in vitro. n=4. All values are Mean±SD. Significant change is indicated by **P<0.01.

FIGS. 4L-4M show an image of a gel (4A) and a graph (4B) showing the results of experiments in which RPE explants from WT and Cryba1 KO mice, along with Sting KO and Sting^{GT} mutant mice, which are animal models with global knockout of STING, were cultured in presence of FAC, Ad-LCN-2, or both with and without ChQ. Western blot analysis and densitometry showed increase in NLRP3, cGAS, and STING expression in the Cryba1 KO RPE cells treated with FAC and Ad-LCN-2; similar treatment along with ChQ addition to block lysosomal function in Sting KO or Sting^{GT} mutant RPE cells did not show an increase in these proteins. n=4. All values are Mean±SD. Significant changes are indicated by **P<0.01.

FIGS. 5A-5B show an image of a gel (5A) and a graph (5B) showing the results of a Western blot analysis and densitometry. The results showed increased levels of redox sensitive protein, SOD in the RPE cells of aged (10 months old) Cryba1 cKO mice, compared to age-matched controls. n=3. All values are Mean±SD. Significant change is indicated by **P<0.01.

FIG. 5C is a graph showing elevated levels of reactive oxygen species (ROS) in aged (10 months old) Cryba1 cKO mice, compared to age-matched controls. n=4. All values are Mean±SD. Significant change is indicated by **P<0.01.

FIGS. 5D-5E is an image of a gel (5D) and a graph (5E) showing the results of Western blot analysis. The results showed that treatment with deferoxamine (Def; 10 mM for 24 h), an iron chelator and STING inhibitor (H-151; 1 mM for 12 h) rescued the levels of NLRP3 inflammasome expression in Cryba1 KO RPE cells treated with FAC and Ad-LCN-2 in vitro. n=4. All values are Mean±SD. Significant change is indicated by **P<0.01.

FIGS. 5F-5G is an image of a gel (5F) and a graph (5G) showing the results of Western blot and densitometry. The results showed increased levels of ferroptosis marker protein FTH1 in the RPE cells of aged (10 months old) Cryba1 cKO mice, compared to age-matched controls. n=4. All values are Mean±SD. Significant change is indicated by **P<0.01.

FIGS. 5H-5I are graphs showing decreased levels of GPX4 activity (5H) and increase in malondialdehyde (MDA) levels (5I), which are markers of ferroptosis activation in cells observed in the RPE cells of aged (10 months old) Cryba1 cKO mice, compared to age-matched controls. n=4. All values are Mean±SD. Significant change is indicated by **P<0.01.

FIG. 5J is a graph showing the results of live cell imaging performed in ARPE19 cells loaded with the fluorescent indicator, liperfluo, which recognizes lipid peroxides that are markers for ferroptosis activation. The results showed higher iperfluo emission intensity in RSL3 (positive control) and FAC+Ad-LCN-2+Chloroquine (FLC) treated cells compared to control. Ferroptosis inhibitor ferrostatin-1 (25 mM; 12 h) abrogated the changes in the FLC-treated cells. n=2. All values are Mean±SD.

FIG. 5K is a graph showing the results of live cell imaging performed in ARPE19 cells loaded with liperfluo dye. The results showed an increase in liperfluo fluorescent emissions in FLC or FLC+IgG treated cells compared to control. However, treatment with the monoclonal antibody targeting the LCN-2 (1 mg/ml for 24 h) could reduce such changes in the FLC-treated cells. n=2. All values are Mean±SD.

FIG. 5L is a graph showing the results of an experiment in which ROS levels were rescued in Def and STING inhibitor-treated cultured Cryba1 KO RPE explants that were exposed to FAC and Ad-LCN-2, compared to vehicle treated group. n=4. All values are Mean±SD or Mean±SEM (for the live cell imaging assay). Significant changes are indicated by *P<0.05, **P<0.01.

FIGS. 6A-6C are graphs showing the results of electroretinography (ERG) studies. The results showed how sub-retinal treatment with mLCN-2 antibody (Clone #6 mAb; 1 mg/ml for 2.5 months) in the Cryba1 cKO mouse model compared to the contralateral eyes that were injected with PBS (6C). It was observed that both the scotopic a-(6B) and b- (6C) wave response were significantly rescued in the monoclonal antibody-treated eyes of the same animals. n=4. All values are Mean±SD.

FIG. 6D shows an image of a gel (top) and a graph (bottom) showing the results of a Western blot analysis. The results showed that subretinal injection with mLCN-2 antibody (Clone #6; 1 mg/ml for 2.5 months) in the Cryba1 cKO mouse model showed a decrease in the expression of autophagosome marker P62/SQSTM1, compared to the contralateral eyes that were injected with PBS. n=4. All values are Mean±SD.

FIGS. 6E-6F are graphs showing that mLCN-2 antibody (Clone #6) sub-retinal treatment of 5 month old Cryba1 cKO mice for 2.5 months also significantly improved the RPE levels levels of GPX4 activity (6E) and MDA (6F), compared to the PBS-treated mice. All values are Mean±SD. Significant change is indicated by **P<0.01. n=4.

FIGS. 7A-7B are general schematics of the light chain sequence (A) and heavy chain sequence (B) of the mLCN-2 antibody (Clone #6).

FIGS. 8A-8B show an image of a gel (8A) and a graph (8B) showing the results of a Western blot analysis. The results showed that subretinal injection with mLCN-2 antibody (Clone #9; 1 mg/ml for 2.5 months) in the Cryba1 cKO mouse model showed a decrease in the expression of autophagosome marker P62/SQSTM1, compared to the contralateral eyes that were injected with PBS. n=4. All values are Mean±SD. Significant change between the groups is denoted as *P<0.05 and **P<0.01.

FIGS. 9A-9B are graphs showing the results of ERG studies. The results showed how sub-retinal treatment with mLCN-2 antibody (Clone #9 mAb; 1 mg/ml for 2.5 months) in the Cryba1 cKO mouse model compared to the contralateral eyes that were injected with PBS. It was observed that both the scotopic a- (9A) and b- (9B) wave response were significantly rescued in the monoclonal antibody-treated eyes of the same animals. n=4. All values are Mean±SD.

FIG. 10A is an image of a gel and a graph showing that in AMD patients, the LCN-2 homodimer variant is prevalent and pathogenic, in accordance with an aspect of the invention. Native PAGE followed by Western blot showed an increased in homodimer:monomer ratio of LCN2 in the RPE cells from human AMD donors compared to age-matched controls. n=3.

FIG. 10B is a schematic showing the different regions of the monoclonal antibody: V_{H}, C_{H}: Variable region heavy chain, Constant region heavy chain; V_{L}, C_{L}: Variable region light chain, Constant region light chain; CDR: Complementarity-determining regions; and FR: Framework region.

FIGS. 11A-11D show that LCN-2 monoclonal antibodies rescue lysosomal function, ferroptosis, autophagosome accumulation and retinal function, in accordance with an aspect of the invention. FIG. 11A is a graph showing sub-retinal injection of the clone #6 mAb to 7.5-month-old Cryba1 cKO mice for 2.5 months also significantly improved the RPE levels of glutathione peroxidase activity, compared to the RPE harvested from the PBS-treated contralateral eye of the same mouse. All values are Mean±SD. Significant changes are indicated by *P<0.05, **P<0.01.

FIG. 11B is an image of a gel and a graph showing the results of a Western blot analysis showing that subretinal injection with clone #6 mAb in the Cryba1 cKO mouse model decreased expression of the autophagosome marker SQSTM1, compared to contralateral eyes that were injected with PBS. n=4. All values are Mean±SD. Significant changes are indicated by *P<0.05, **P<0.01.

FIG. 11C is a set of images from cell imaging in ARPE19 cells loaded with liperfluo dye showing increased liperfluo fluorescent emissions in FLC or FLC+IgG (mouse immunoglobulin, 1 μg/ml for 1 h) treated cells, compared to control. However, treatment with the monoclonal antibody (clone #6 mAb) targeting LCN2 (1 μg/ml for 1 h) could reduce such changes in the FLC-treated cells. Scale bar: 50 μm. All values are Mean±SD or Mean±SEM (for the live cell imaging assay). n=2. Significant changes are indicated by *P<0.05, **P<0.01.

FIG. 11D is a set of graphs showing scotopic a- (left) and b-wave (right) responses measured at three different light intensities (0.01, 0.1 and 1 cd*s/m2), suggesting alterations in retinal function in the LCN2 overexpressing SM injected NOD-SCID mice, which was rescued partially, only after exposure to the highest light intensity upon treatment with clone #6 mAb. n=4.

FIG. 12 is a schematic showing that CN2 deregulates the autophagy process in RPE cells and that targeting LCN2 variants with a monoclonal antibody can significantly diminish an AMD-like phenotype in a mouse model. LCN2 upregulation in RPE cells occurs through the activation of the NFκB-STAT1 signaling axis both in the cryba1 cKO mouse model and in human AMD donor samples. Here it is shown that LCN2 binds to and regulates the activity of ATG4B by forming a complex with ATG4B and LC3, thereby modulating autophagosome processing (autophagy flux). LCN2 monomer and dimer variants are upregulated in RPE cells from the cryba1 cKO mouse model and human dry AMD donors, while the homodimer variant, the only form secreted from the RPE cells, can trigger retinal degeneration. Further, the homodimer variant is unable to form the complex with ATG4B and LC3. Alterations in autophagosome processing due to LCN2 upregulation accompanied with abnormal lysosomal function in RPE cells trigger abnormal iron accumulation. Alterations in iron homeostasis in RPE cells trigger inflammasome activation through the upregulation of the CGAS-STING1 pathway leading to oxidative stress and ferroptosis. Targeting LCN2 with a monoclonal antibody (clone #6 mAb) that recognizes and neutralizes both LCN2 variants can mitigate the changes in autophagy, lipid peroxidation or ferroptosis and diminish the AMD-like phenotype in a mouse model.

### DETAILED DESCRIPTION OF THE INVENTION

It has been discovered that monoclonal antibodies directed against human LCN-2 that neutralize both human LCN-2 monomer and human LCN-2 homodimer may provide any one or more of the following in a dry AMD-like mouse model: increased autophagy in RPE cells, reduced ferroptotic death of RPE cells, reduced lipid peroxidation in RPE cells, reduced inflammasome activation in RPE cells, increased glutathione peroxidase activity in RPE cells, and improved retinal function.

An aspect of the invention provides a monoclonal antibody, or an antigen binding portion thereof, which neutralizes human Lipocalin 2 (LCN-2). LCN-2 is a member of the adipokine family of proteins. LCN-2 exists as either a monomer or a homodimer (Santiago-Sánchez et al., Int. J. Mol. Sci., 21(12): 4365 (2020); Barasch et al., Nat. Commun., 7: 12973 (2016)). In an aspect of the invention, the antibodies and antigen binding portions thereof neutralize one or both of human LCN-2 monomer and human LCN-2 homodimer. Human LCN-2 consists of the amino acid sequence of

(SEQ ID NO: 1)
MPLGLLWLGLALLGALHAQAQDSTSDLIPAPPLSKVPLQQNFQDNQFQGK
WYVVGLAGNAILREDKDPQKMYATIYELKEDKSYNVTSVLFRKKKCDYWI
RTFVPGCQPGEFTLGNIKSYPGLTSYLVRVVSTNYNQHAMVFFKKVSQNR
EYFKITLYGRTKELTSELKENFIRFSKSLGLPENHIVFPVPIDQCIDG.

The term “neutralize” (and words stemming therefrom), when used to refer to the inventive antibodies, or antigen binding portions thereof, which neutralize LCN-2 (monomer or homodimer), is meant that the antibodies, or antigen binding portions thereof, inhibit one or more of the biological activities associated with increased LCN-2 (monomer or homodimer, respectively) in the RPE that contribute to the AMD-like phenotype in a mouse model. These biological activities associated with increased LCN-2 (monomer or homodimer, respectively) in the RPE may include, for example, any one or more of decreased autophagy in RPE cells, increased ferroptotic death of RPE cells, increased lipid peroxidation in RPE cells, increased inflammasome activation in RPE cells, and reduced glutathione peroxidase activity in RPE cells.

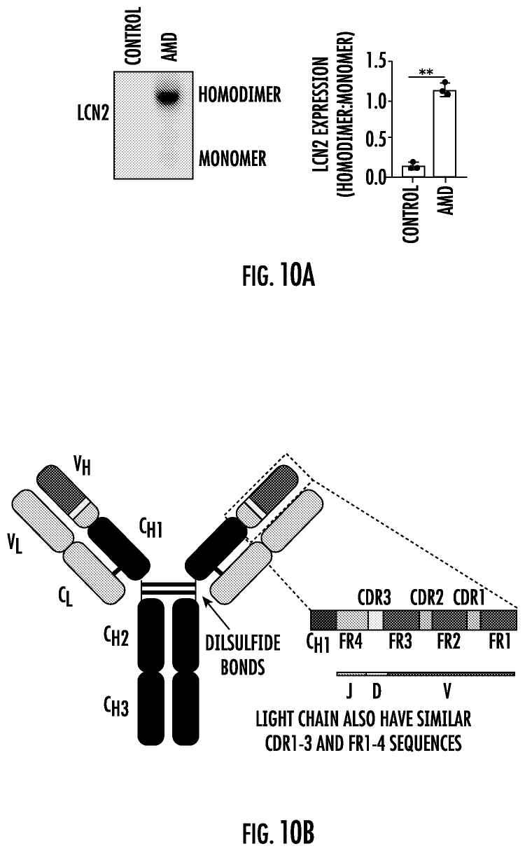

An antibody typically consists of four polypeptides: two identical copies of a heavy (H) chain polypeptide and two identical copies of a light (L) chain polypeptide (see FIG. 10B). Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have the same general structure, with each region comprising four framework regions, whose sequences are relatively conserved. The four heavy chain framework regions (VH FRs) are referred to as VH FR1, VH FR2, VH FR3, and VH FR4. The four light chain framework regions (VL FRs) are referred to as VL FR1, VL FR2, VL FR3, and VL FR4. The framework regions are connected by three complementarity determining regions (CDRs). The three CDRs, known as CDR1, CDR2, and CDR3, form the “hypervariable region” of an antibody, which is responsible for antigen binding.

In an aspect of the invention, the antibody, or antigen binding portion thereof, comprises two polypeptide chains, each of which comprises a variable region comprising a complementarity determining region (CDR) 1, a CDR2, and a CDR3 of an antibody. Preferably, the first polypeptide chain comprises a heavy chain CDR1, a heavy chain CDR2, a heavy chain CDR3, and the second polypeptide chain comprises a light chain CDR1, a light chain CDR2, and a light chain CDR3. The CDR binding sequences may be determined by methods known in the art such as, for example, the methodology of the international ImMunoGeneTics information system (IMGT) or Kabat (Wu and Kabat, J Exp. Med., 132: 211-250 (1970)).

An aspect of the invention provides an antibody, or antigen binding portion thereof, which neutralizes human LCN-2, wherein the antibody, or antigen binding portion thereof comprises:
(A) the heavy chain complementary determining region (VH CDR) 1 amino acid sequence of SEQ ID NO: 7;
the VH CDR2 amino acid sequence of SEQ ID NO: 9;
the VH CDR3 amino acid sequence of SEQ ID NO: 11;
the light chain complementary determining region (VL CDR) 1 amino acid sequence of SEQ ID NO: 14;
the VL CDR2 amino acid sequence of (LVS) SEQ ID NO: 16; and
the VL CDR3 amino acid sequence of SEQ ID NO: 18; or
(B) the VH CDR1 amino acid sequence of SEQ ID NO: 23;
the VH CDR2 amino acid sequence of (GTN) SEQ ID NO: 25;
the VH CDR3 amino acid sequence of SEQ ID NO: 27;
the VL CDR1 amino acid sequence of SEQ ID NO: 30;
the VL CDR2 amino acid sequence of SEQ ID NO: 32; and
the VL CDR3 amino acid sequence of SEQ ID NO: 34.

In an aspect of the invention, the antibody, or antigen binding portion thereof, comprises the framework regions of each of the heavy and light chains, in addition to the CDRs of the heavy and light chains. In this regard, the antibody, or antigen binding portion thereof, may comprise:
(A) the VH FR1 amino acid sequence of SEQ ID NO: 6;
the VH CDR1 amino acid sequence of SEQ ID NO: 7;
the VH FR2 amino acid sequence of SEQ ID NO: 8;
the VH CDR2 amino acid sequence of SEQ ID NO: 9;
the VH FR3 amino acid sequence of SEQ ID NO: 10;
the VH CDR3 amino acid sequence of SEQ ID NO: 11;
the VH FR4 amino acid sequence of SEQ ID NO: 12;
the VL FR1 amino acid sequence of SEQ ID NO: 13;
the VL CDR1 amino acid sequence of SEQ ID NO: 14;
the VL FR2 amino acid sequence of SEQ ID NO: 15;
the VL CDR2 amino acid sequence of (LVS) SEQ ID NO: 16;
the VL FR3 amino acid sequence of SEQ ID NO: 17;
the VL CDR3 amino acid sequence of SEQ ID NO: 18; and
the VL FR4 amino acid sequence of SEQ ID NO: 19; or
(B) the VH FR1 amino acid sequence of SEQ ID NO: 22;
the VH CDR1 amino acid sequence of SEQ ID NO: 23;
the VH FR2 amino acid sequence of SEQ ID NO: 24;
the VH CDR2 amino acid sequence of (GTN) SEQ ID NO: 25;
the VH FR3 amino acid sequence of SEQ ID NO: 26;
the VH CDR3 amino acid sequence of SEQ ID NO: 27;
the VH FR4 amino acid sequence of SEQ ID NO: 28;
the VL FR1 amino acid sequence of SEQ ID NO: 29;
the VL CDR1 amino acid sequence of SEQ ID NO: 30;
the VL FR2 amino acid sequence of SEQ ID NO: 31;
the VL CDR2 amino acid sequence of SEQ ID NO: 32;
the VL FR3 amino acid sequence of SEQ ID NO: 33;
the VL CDR3 amino acid sequence of SEQ ID NO: 34 and
the VL FR4 amino acid sequence of SEQ ID NO: 35.

In an aspect of the invention, the antibody, or antigen binding portion thereof, may comprise a full length VH amino acid sequence comprising the VH CDRs and VH FRs described above and/or a full-length VL amino acid sequence comprising the VL CDRs and VL FRs described above. In this regard, the antibody, or antigen binding portion thereof, may comprise:
(A) the heavy chain variable region (VH) amino acid sequence of SEQ ID NO: 20 and the light chain variable region (VL) amino acid sequence of SEQ ID NO: 21; or
(B) the VH amino acid sequence of SEQ ID NO: 36 and the VL amino acid sequence of SEQ ID NO: 37.

In an aspect of the invention, the antibody, or antigen binding portion thereof, can comprise an amino acid sequence of a variable region of an antibody comprising the CDRs set forth above. In this regard, the antibody, or antigen binding portion thereof, can comprise, consist of, or consist essentially of, the variable region of a heavy chain, the variable region of a light chain, or both the variable region of a heavy chain and the variable region of a light chain.

In an aspect of the invention, the antibody, or antigen binding portion thereof, can further comprise a constant region of an antibody. In this regard, the antibody, or antigen binding portion thereof, can further comprise the constant region of a heavy chain, or the constant region of a light chain, or both the constant region of a heavy chain and the constant region of a light chain.

The antibody of the invention can be any type of immunoglobulin that is known in the art. For instance, the antibody may be a recombinant antibody. The antibody may be of any isotype, e.g., IgA, IgD, IgE, IgG (e.g., IgG1, IgG2, IgG3, or IgG4), IgM, etc. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody may be a polyclonal antibody or a monoclonal antibody. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen.

Methods of testing antibodies for the ability to bind to LCN-2 are known in the art and include any antibody-antigen binding assay, such as, for example, radioimmunoassay (RIA), enzyme-linked immunosorbent assay (ELISA), Western blot, immunoprecipitation, and competitive inhibition assays. Methods of testing antibodies for the ability to neutralize to LCN-2 are known in the art and include, for example, the assays described in the Examples.

Suitable methods of making antibodies are known in the art and include, for example, standard hybridoma methods, Epstein-Barr virus (EBV)-hybridoma methods, and bacteriophage vector expression systems. Antibodies may be produced in non-human animals such as, for example, rabbits.

The terms “fragment of an antibody,” “antibody fragment,” “functional fragment of an antibody,” and “antigen binding portion” are used interchangeably herein to mean one or more fragments or portions of an antibody that retain the ability to bind to an antigen. The antigen binding portion of the antibody comprises, for example, one or more CDRs, the variable region (or portions thereof), the constant region (or portions thereof), or combinations thereof. In an aspect of the invention, the antigen binding portion of the antibody comprises the heavy chain CDR1, heavy chain CDR2, heavy chain CDR3, light chain CDR1, light chain CDR2, and light chain CDR3. In an aspect of the invention, the antigen binding portion of the antibody comprises the heavy chain FR1, heavy chain CDR1, heavy chain FR2, heavy chain CDR2, heavy chain FR3, heavy chain CDR3, heavy chain FR4, light chain FR1, light chain CDR1, light chain FR2, light chain CDR2, light chain FR3, light chain CDR3, and light chain FR4. In an aspect of the invention, the antigen binding portion of the antibody comprises the heavy chain variable region and the light chain variable region.

An aspect of the invention provides an antigen-binding portion of the antibody. The antigen-binding portion of the antibody can be, for example, a Fab fragment (Fab), F(ab′)2 fragment, Fab′ fragment, FV fragment, scFv, diabody, triabody, tetrabody, or minibody. A Fab fragment is a monovalent fragment consisting of the VL, VH, CL, and CH1 domains. A F(ab′)2 fragment is a bivalent fragment comprising two Fab fragments linked by a disulfide bridge at the hinge region. An Fv fragment consists of the VL and VH domains of a single arm of an antibody. A single chain Fv (scFv) is a monovalent molecule consisting of the two domains of the Fv fragment (i.e., VL and VH) joined by a synthetic linker which enables the two domains to be synthesized as a single polypeptide chain. A diabody is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites.

Further provided by an aspect of the invention is a nucleic acid comprising a nucleotide sequence encoding any of the antibodies, or antigen binding portions thereof, described herein with respect to other aspects of the invention. “Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. The nucleic acids of an aspect of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

In an aspect, the nucleic acids of the invention can be incorporated into a recombinant expression vector. In this regard, an aspect of the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an aspect, the recombinant expression vector of the invention can be any suitable recombinant expression vector, and can be used to transform or transfect any suitable host cell. Suitable vectors include those designed for propagation and expansion or for expression or both, such as plasmids and viruses.

In an aspect, the recombinant expression vectors of the invention can be prepared using standard recombinant DNA techniques. Constructs of expression vectors, which are circular or linear, can be prepared to contain a replication system functional in a prokaryotic or eukaryotic host cell. Replication systems can be derived, e.g., from ColEl, 2 μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can comprise a native or nonnative promoter operably linked to the nucleotide sequence encoding the inventive antibody or antigen binding portion thereof. The selection of promoters, e.g., strong, weak, inducible, tissue-specific and developmental-specific, is within the ordinary skill of the artisan. Similarly, the combining of a nucleotide sequence with a promoter is also within the ordinary skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

An aspect of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant antibody or antigen binding portion thereof, the host cell may be a mammalian cell. The host cell may be a human cell.

Also provided by an aspect of the invention is a population of cells comprising at least one host cell described herein. The population of cells can be a heterogeneous population comprising the host cell comprising any of the recombinant expression vectors described, in addition to at least one other cell which does not comprise any of the recombinant expression vectors. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

The antibodies and antigen binding portions thereof, nucleic acids, recombinant expression vectors, host cells (including populations thereof), all of which are collectively referred to as “inventive anti-LCN-2 materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) antibody preparation is one in which the antibody is purer than antibodies in their natural environment within the body. Such antibodies may be produced, for example, by standard purification techniques. In some aspects, a preparation of an antibody is purified such that the antibody represents at least about 50%, for example, at least about 70%, of the total antibody content of the preparation. For example, the purity can be at least about 50%, can be greater than about 60%, about 70% or about 80%, about 90%, about 95%, or can be about 100%.

The inventive anti-LCN-2 materials can be formulated as a composition (e.g., pharmaceutical composition) comprising any of the inventive antibodies, antigen binding portions thereof, nucleic acids, recombinant expression vectors, host cells (including populations thereof) and a carrier (e.g., a pharmaceutically or physiologically acceptable carrier). Furthermore, the inventive anti-LCN-2 materials can be used in the methods described herein alone or as part of a pharmaceutical formulation.

The composition (e.g., pharmaceutical composition) can comprise more than one inventive anti-LCN-2 material. Alternatively, or in addition, the composition can comprise one or more other pharmaceutically active agents or drugs. Examples of such other pharmaceutically active agents or drugs that may be suitable for use in the pharmaceutical composition include lampalizumab (anti-complement factor D; Genentech) for patients with geographic atrophy secondary to AMD); brolicizumab (pan-isoform ant-VEGF-A; Novartis) for wet AMD; OPT-302 (soluble VEGF-C/D receptor; Ophthea) for wet AMD; PanOptica's topical VEGF inhibitor for wet AMD; pegpleranib (DNA aptamer binding to PDGF isoforms; Ophtotech/Novartis) optionally combined with LUCENTIS™ (ranibizumab injection); rinucumab (anti-PDGF receptor; Regeneron) optionally co-formulated with EYLEA™ (aflibercept); DE-120 (anti-PDGF/VEGF bispecific; Santen); vorolanib (oral RTK inhibitor that inhibits kinase activity for pDGF and VEGF; Tyrogenex); nevacumab (anti-angiopoeitin 2; Regeneron) optionally combined with EYLEA™ (aflibercept); RG-7716 (anti-angiopoeitin 2/VEGF bispecific; Chugai); ARP-1536 (anti VE PTP; Akebia/Aeripo); ICON-1 (chimeric protein binding to Tissue Factor; Iconic Therapeutic); and carotuximab (anti-endogin; Tracon/Santen).

The carrier can be any of those conventionally used and is limited only by physio-chemical considerations, such as solubility and lack of reactivity with the active compound(s) and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular inventive anti-LCN-2 material and other active agents or drugs used, as well as by the particular method used to administer the inventive anti-LCN-2 material.

Any suitable dose of the inventive anti-LCN-2 materials, or composition thereof, can be administered to a subject. The appropriate dose will vary depending upon such factors as the subject's age, weight, height, sex, general medical condition, previous medical history, and disease progression, and can be determined by a clinician. The amount or dose should be sufficient to effect the desired biological response, e.g., a therapeutic or prophylactic response, in the subject over a clinically reasonable time frame.

For purposes of the invention, an assay, which comprises, for example, comparing the extent to which LCN-2 is neutralized in the RPE cells upon administration of a given dose of the inventive anti-LCN-2 material to a subject, among a set of subject of which is each given a different dose of the inventive anti-LCN-2 material, could be used to determine a starting dose to be administered to a subject. The extent to which LCN-2 is neutralized in the RPE cells upon administration of a certain dose can be assayed by methods known in the art.

The inventive anti-LCN-2 materials, or composition thereof, can be administered to the subject by various routes including, but not limited to, topical, subcutaneous, intramuscular, intradermal, intraperitoneal, intrathecal, intravenous, subretinal injection, and intravitreal injection. In one aspect, the inventive anti-LCN-2 material, or composition thereof, can be directly administered (e.g., locally administered) by direct injection into the eye by subretinal or inravitreal injection or by topical application (e.g., as eye drops). When multiple administrations are given, the administrations can be at one or more sites in a subject and a single dose can be administered by dividing the single dose into equal portions for administration at one, two, three, four or more sites on the individual.

An aspect of the invention provides a method of reducing or preventing ferroptotic death of RPE cells in a subject in need thereof, the method comprising administering any of the inventive anti-LCN-2 materials, or a composition thereof, to the subject.

Another aspect of the invention provides a method of reducing or preventing one or both of lipid peroxidation and inflammasome activation in RPE cells in a subject in need thereof, the method comprising administering any of the inventive anti-LCN-2 materials, or a composition thereof, to the subject.

An aspect of the invention provides a method of reducing or preventing a decrease in one or both of scotopic a- and b-wave responses in a retina of a subject in need thereof, the method comprising administering any of the inventive anti-LCN-2 materials, or a composition thereof, to the subject, wherein the scotopic a- and b-wave responses in the retina are measured by electroretinography (ERG).

An aspect of the invention provides a method of increasing one or both of scotopic a- and b-wave responses in a retina of a subject in need thereof, the method comprising administering any of the inventive anti-LCN-2 materials, or a composition thereof, to the subject, wherein the scotopic a- and b-wave responses in the retina are measured by electroretinography (ERG).

An aspect of the invention provides a method of increasing one or both of autophagy and glutathione peroxidase activity in RPE cells in a subject in need thereof, the method comprising administering any of the inventive anti-LCN-2 materials, or a composition thereof, to the subject.

In one aspect, the subject has AMD or is at risk for developing AMD. The AMD can be wet AMD or atrophic (dry) AMD. The subject may have geographic atrophy secondary to AMD. As such, an aspect of the invention also provides a method of treating or preventing AMD in a subject in need thereof, the method comprising administering any of the inventive anti-LCN-2 materials, or a composition thereof, to the subject.

In one aspect, the subject has Stargardt's macular retinal degeneration or is at risk for developing Stargardt's macular retinal degeneration. LCN-2 upregulation is also seen in an animal model of Stargardt's disease (Parmar et al., Invest. Ophthalmol. Vis. Sci., 57(7): 3257-67 (2016)). Accordingly, an aspect of the invention also provides a method of treating or preventing Stargardt's macular retinal degeneration.

Administration of the inventive anti-LCN-2 materials, or composition thereof, can be “prophylactic” or “therapeutic.” When provided prophylactically, the inventive anti-LCN-2 material, or composition thereof, is provided in advance of a subject's diagnosis with AMD. For example, subjects at risk for developing AMD or Stargardt's macular retinal degeneration are a preferred group of patients treated prophylactically. The prophylactic administration of the inventive anti-LCN-2 materials, or composition thereof, thereof prevents, ameliorates, or delays AMD or Stargardt's macular retinal degeneration. When provided therapeutically, the inventive anti-LCN-2 material or composition thereof is provided at or after the diagnosis of AMD or Stargardt's macular retinal degeneration.

When the subject has already been diagnosed with AMD or Stargardt's macular retinal degeneration, the inventive anti-LCN-2 materials or composition thereof can be administered in conjunction with other therapeutic treatments such as lampalizumab (anti-complement factor D; Genentech); brolicizumab (pan-isoform ant-VEGF-A; Novartis); OPT-302 (soluble VEGF-C/D receptor; Ophthea); PanOptica's topical VEGF inhibitor; pegpleranib (DNA aptamer binding to PDGF isoforms; Ophtotech/Novartis); LUCENTIS™ (ranibizumab injection); rinucumab (anti-PDGF receptor; Regeneron); EYLEA™ (aflibercept); DE-120 (anti-PDGF/VEGF bispecific; Santen); vorolanib (oral RTK inhibitor that inhibits kinase activity for pDGF and VEGF; Tyrogenex); nevacumab (anti-angiopoeitin 2; Regeneron); RG-7716 (anti-angiopoeitin 2/VEGF bispecific; Chugai); ARP-1536 (anti VE PTP; Akebia/Aeripo); ICON-1 (chimeric protein binding to Tissue Factor; Iconic Therapeutic); and/or carotuximab (anti-endogin; Tracon/Santen).

The terms “treat,” and “prevent,” as used herein, do not necessarily imply 100% or complete treatment or prevention. Rather, there are varying degrees of treatment or prevention of which a person of ordinary skill in the art recognizes as having a potential benefit or therapeutic effect.

There are a variety of suitable formulations of the pharmaceutical composition for the inventive methods. Any suitable carrier can be used within the context of the invention, and such carriers are well known in the art. The choice of carrier will be determined, in part, by the particular site to which the composition is to be administered (e.g., ocular cells, RPE cells, photoreceptor cells, rods, and cones) and the particular method used to administer the composition. The pharmaceutical composition can optionally be sterile or sterile.

Suitable formulations for the pharmaceutical composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the pharmaceutical composition for use in the inventive method is formulated to protect the inventive anti-LCN-2 material, or composition thereof, from damage prior to administration. For example, the pharmaceutical composition can be formulated to reduce loss of the inventive anti-LCN-2 material, or composition thereof, on devices used to prepare, store, or administer the inventive anti-LCN-2 material, or composition thereof, such as glassware, syringes, or needles. The pharmaceutical composition can be formulated to decrease the light sensitivity and/or temperature sensitivity of the inventive anti-LCN-2 material, or composition thereof. To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition may extend the shelf life of the inventive anti-LCN-2 material, or composition thereof, facilitate administration, and increase the efficiency of the inventive method.

Aspects, including embodiments, of the subject matter described herein may be beneficial alone or in combination, with one or more other aspects or embodiments. Without limiting the foregoing description, certain non-limiting aspects of the disclosure numbered 1-21 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) An isolated or purified monoclonal antibody, or an antigen binding portion thereof, which neutralizes human Lipocalin 2 (LCN-2).

(2) The antibody, or antigen binding portion thereof, of aspect 1, wherein the antibody and antigen binding portion thereof neutralize one or both of human LCN-2 monomer and human LCN-2 homodimer.

(3) The antibody, or antigen binding portion thereof, of aspect 1 or 2, wherein human LCN-2 consists of the amino acid sequence of:

(SEQ ID NO: 1)
MPLGLLWLGLALLGALHAQAQDSTSDLIPAPPLSKVPLQQNFQDNQFQGK
WYVVGLAGNAILREDKDPQKMYATIYELKEDKSYNVTSVLFRKKKCDYWI
RTFVPGCQPGEFTLGNIKSYPGLTSYLVRVVSTNYNQHAMVFFKKVSQNR
EYFKITLYGRTKELTSELKENFIRFSKSLGLPENHIVFPVPIDQCIDG.

(4) The antibody, or antigen-binding portion of the antibody of aspect 1 or 2, wherein the antigen-binding portion is a Fab fragment (Fab), F(ab′)2 fragment, Fab′ fragment, Fv fragment, scFv, diabody, triabody, tetrabody, or minibody.

(5) The antibody, or antigen binding portion thereof, of aspect 1 or 2 comprising:
(A) the heavy chain complementary determining region (VH CDR) 1 amino acid sequence of SEQ ID NO: 7;
the VH CDR2 amino acid sequence of SEQ ID NO: 9;
the VH CDR3 amino acid sequence of SEQ ID NO: 11;
the light chain complementary determining region (VL CDR) 1 amino acid sequence of SEQ ID NO: 14;
the VL CDR2 amino acid sequence of (LVS) SEQ ID NO: 16; and
the VL CDR3 amino acid sequence of SEQ ID NO: 18; or
(B) the VH CDR1 amino acid sequence of SEQ ID NO: 23;
the VH CDR2 amino acid sequence of (GTN) SEQ ID NO: 25;
the VH CDR3 amino acid sequence of SEQ ID NO: 27;
the VL CDR1 amino acid sequence of SEQ ID NO: 30;
the VL CDR2 amino acid sequence of SEQ ID NO: 32; and
the VL CDR3 amino acid sequence of SEQ ID NO: 34.

(6) The antibody, or antigen binding portion thereof, of aspect 1 or 2 comprising:
(A) the heavy chain variable region (VH) amino acid sequence of SEQ ID NO: 20 and the light chain variable region (VL) amino acid sequence of SEQ ID NO: 21; or
(B) the VH amino acid sequence of SEQ ID NO: 36 and the VL amino acid sequence of SEQ ID NO: 37.

(7) A nucleic acid comprising a nucleotide sequence encoding the antibody, or antigen binding portion thereof, of aspect 1 or 2.

(8) A recombinant expression vector comprising the nucleic acid of aspect 7.

(9) A host cell comprising the recombinant expression vector of aspect 8.

(10) A population of host cells comprising at least two host cells of aspect 9.

(11) A pharmaceutical composition comprising:
the antibody, or the antigen-binding portion of the antibody, of aspect 1 or 2,
a nucleic acid comprising a nucleotide sequence encoding the antibody, or antigen binding portion thereof,
a recombinant expression vector comprising the nucleic acid,
a host cell comprising the recombinant expression vector,
or a population of host cells comprising at least two of the host cells,
and a pharmaceutically acceptable carrier.

(12) A method of reducing or preventing ferroptotic death of retinal pigmented epithelium (RPE) cells in a subject in need thereof, the method comprising administering:
the antibody, or the antigen-binding portion of the antibody, of aspect 1 or 2,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject.

(13) A method of reducing or preventing one or both of lipid peroxidation and inflammasome activation in retinal pigmented epithelium (RPE) cells in a subject in need thereof, the method comprising administering:
the antibody, or the antigen-binding portion of the antibody, of aspect 1 or 2,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier, to the subject.

(14) A method of reducing or preventing a decrease in one or both of scotopic a- and b-wave responses in a retina of a subject in need thereof, the method comprising administering:
the antibody, or the antigen-binding portion of the antibody, of aspect 1 or 2,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject, wherein the scotopic a- and b-wave responses in the retina are measured by electroretinography (ERG).

(15) A method of increasing one or both of scotopic a- and b-wave responses in a retina of a subject in need thereof, the method comprising administering:
the antibody, or the antigen-binding portion of the antibody, of aspect 1 or 2,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject, wherein the scotopic a- and b-wave responses in the retina are measured by electroretinography (ERG).

(16) A method of increasing one or both of autophagy and glutathione peroxidase activity in RPE cells in a subject in need thereof, the method comprising administering:
the antibody, or the antigen-binding portion of the antibody, of aspect 1 or 2,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject.

(17) The method of aspect 12, wherein the subject has age-related macular degeneration (AMD) or is at risk for developing AMD.

(18) A method of treating or preventing age-related macular degeneration (AMD) in a subject in need thereof, the method comprising administering:
the antibody, or the antigen-binding portion of the antibody, of aspect 1 or 2,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject.

(19) The method of aspect 17, wherein the AMD is atrophic AMD, optionally wherein the atrophic AMD includes geographic atrophy.

(20) The method of aspect 17, wherein the AMD is wet AMD.

(21) The method of aspect 12, wherein the antibody, antigen-binding portion of the antibody, nucleic acid, recombinant expression vector, host cell, population of host cells, or pharmaceutical composition is administered by subretinal injection, intravitreal injection, or topical administration.

The following examples further illustrate the invention but, of course, should not be construed as in any way limiting its scope.

### EXAMPLES

The following materials and methods were employed for the experiments described in Examples 1-9.

### Antibodies

The primary antibodies used in this study were: LCN-2 mAb Clone #6 (generated in the inventors' laboratory), ATG4B (Thermo Fisher, 701882), GFP (Cell Signaling Technology, 2555S), GST (Cell Signaling Technology, USA, 2622S), FTH1 (Cell Signaling Technology, 4393S), LC3B (Cell Signaling Technology, 2775S), NLRP3 (Cell Signaling Technology, 15101S), SQSTM1 (Cell Signaling Technology, 5114S), cGAS (Sigma Aldrich, ABF124), STING (Novus Biologicals, NBP2-24683), IL-13 (Abcam, ab9722), CATALASE (Abcam, ab209211), SOD (Cell Signaling Technology, 37385S), VINCULIN (Abcam, ab129002) and ACTIN (Sigma Aldrich, A2066-100UL). The secondary antibodies used in this study were anti-rabbit (KPL, 074-1506) and anti-mouse (KPL, 074-1806).

### Animals

Both male and female PA3/A1-crystallin conditional knockout mice (Cryba1 cKO C57Bl/6J mice) and Cryba1 KO were generated as previously explained (Valapala et al., Autophagy, 10: 480-496 (2014)). Sting knockout (KO), Sting Goldenticket (Sting^{GT}) mutant and NOD-SCID mice (4 week old NOD.CB17-Prkdescid/J mice) were purchased from The Jackson Laboratory, USA. The Sting KO and Sting^{GT} were bred to generate the animals required for this study. All animal studies were conducted in accordance with the Guide for the Care and Use of Animals (National Academy Press) and were approved by the University of Pittsburgh Animal Care and Use Committee.

### RPE Explant Culture

RPE explant culture was done as previously described (Shang et al., Commun. Biol., 4(1): 850 (2021)). Briefly, eyeballs were enucleated from WT, Cryba1 KO, Sting KO or Sting^{GT} mice respectively, immediately after euthanasia by putting in CO₂ chamber. The posterior eyecups were cut into four petals after the removal of anterior segments and the neural retina was carefully removed. The resulting RPE-Choroid-Sclera (RCS) complexes were flattened onto PVDF membranes with the RPE cells facing up and cultured as previously described. For experiments involving iron regulation, RPE explants from different genotypes were cultured in presence of ferric ammonium citrate (FAC, Sigma Aldrich, RES20400-A702X) at a dose of 250 μM for 72 h (Hadziahmetovic et al., Am. J Pathol., 179(1): 335-48 (2011)); Chothe et al., Biochem. Biophys. Res. Commun., 405(2): 244-9 (2011)), Adenovirus-LCN-2 (10⁷ vg/ml, Ad-LCN-2, Vector Biolabs, ADV-263329) construct for 48 h and with or without chloroquine (ChQ; Sigma Aldrich, C6628-25G), a lysosomal blocker at a dose of 50 μM for the last 6 h of the experimental duration. For rescue experiments, Cryba1 KO explants were cultured and pre-treated with either Def (100 μM for 24 h; Sigma Aldrich, D9533-1G) or Sting inhibitor (H-151; 1 μM for 6 hours; Invitrogen, inh-h151) which was followed by FAC and Ad-LCN-2 treatment as explained above.

### Sub-Retinal Injections, Electroretinography and Spectral Domain-Optical Coherence Tomography (SD-OCT)

Sub-retinal injection was performed on 4-week-old NOD-SCID and 5-month-old Cryba1 cKO mice (Ghosh et al., Commun. Biol., 2: 348 (2019)). Mice were anaesthetized and sub-retinal injections of either RPESM from floxed RPE explant culture overexpressing LCN-2 or the same SM pre-treated with 1 μg/ml of the monoclonal antibody (LCN-2 mAb Clone #6) for 1 h, were given to the contralateral eyes of the same NOD-SCID mice, respectively (Ghosh et al., Commun. Biol., 2: 348 (2019); Liddelow et al., Nature, 541(7638): 481-487 (2017)). The Cryba1 cKO mice were also subretinally injected with either PBS in one eye or the monoclonal antibody in contralateral eyes of the same mouse (Ghosh et al., Commun. Biol., 2: 348 (2019)). For the NOD-SCID mice, treatment was done for one month, whereas the Cryba1 cKO mice were treated for 2.5 months. Both the treated and untreated mice were dark adapted for 24 h and then were anaesthetized by intraperitoneal (100 μl) injection of ketamine (50 mg/kg body weight)/xylazine (10 mg/kg body weight) was used for anesthesia, and then subjected to electroretinography to evaluate retinal function by estimating the scotopic a- and b-wave responses using the Celeris Diognosys System, USA (Valapala et al., Autophagy, 10(3): 480-96 (2014)). Responses were measured at three different light intensities (0.01, 0.1 and 1 cd*s/m²). After the experiment, the Cryba1 cKO animals were euthanized with CO₂ gas and the eyes were harvested for further experiments to evaluate the expression of protein levels by Western blot or other biochemical experiments. NOD-SCID mice were also subjected to SD-OCT analysis using a Bioptigen Envisu R2210, USA system. OCT images were analyzed keeping the optic nerve head (ONH) at the 0 position and then the optical sections (100 sections per retina) were measured from each eye ranging from −2.0 to +2.0 mm with respect to the ONH using the FIJI-ImageJ (NIH) plugin provided with the instrument along with Diver 2.4 software (Bioptigen) (Ghosh et al., Commun. Biol., 2: 348 (2019)).

### Co-Immunoprecipitation

Cryba1 floxed RPE flat mount cultures were infected with Adenovirus-LCN-2-GFP (Ad-LCN-2-GFP, Vector Biolabs, 2000) or Adenovirus-GFP constructs (Ad-GFP, Vector Biolabs, 1060) at a dose of 10⁷ vg/ml for 48 h and lysed in RIPA buffer (EMD Milipore, 20-188) supplemented with a protease inhibitor cocktail (Sigma Aldrich, 13786-1ML). The cell lysates were incubated with GFP magnetic beads (Chromotek, gtd-10) for 2 h at 4° C. Collected beads were washed with washing buffer and eluted with 2×SDS-sample buffer as previously explained (Ghosh et al., Commun. Biol., 4(1): 248 (2021)). The eluted samples were analyzed on a SDS-PAGE gel followed by Western blot to assess the binding of ATG4B. The pull-down was confirmed by re-probing the membrane with an anti-GFP antibody (Cell Signaling Technology, 2555S) which showed two distinct molecular weight bands-one for the LCN-2 fused to GFP in cells infected with the Ad-LCN-2-GFP construct and the other for a lower molecular weight band for GFP protein only in cells that were infected with the Ad-GFP construct.

### Immunofluorescence

ARPE19 cells (ATCC, CRL-2302) were grown to 60% confluency as described by the manufacturer. The cells were infected with an Ad-LCN-2-GFP construct (mentioned above) (10⁷ vg/ml) for 48 h, where the media was changed after 24 h of infection. The cells were fixed with 2% paraformaldehyde (PFA, Alfa Aesar, J61899-AP) for 30 mins at 4° C., then permeabilized and blocked with 5% Donkey serum (Sigma Aldrich, D9663-10ML) in 1×PBS containing 0.1% Triton-X100 (Sigma Aldrich, T8787-250ML) for 30 min at room temperature (RT). The cells were washed twice with PBS and then stained with anti-ATG4B antibody (Thermo Fisher, 701882) diluted to 1:100 in the blocking buffer without Triton-X100 overnight in 4° C. The cells were then washed with PBS+0.1% Tween 20 (PBST, Sigma Aldrich, P7949-100ML) thrice and incubated in 1 μM DAPI (Southern Biotech, 0100-20), to stain the nucleus for 10 min at RT. The cells were again washed with PBST five times and visualized on an Olympus IX81 confocal microscope. Analysis of co-localization between LCN-2 (Green) and ATG4B (Red) was done using the JACoP plugin of ImageJ software and the Pearson's co-efficient was calculated to quantify the level of co-localization between the two proteins as previously described (Bolte et al., J. Microsc., 224(Pt 3): 213-32 (2006)).

### Thermal Shift Assay

Changes in melting temperature (Tm) was assessed to evaluate the binding propensity of LCN-2 and ATG4B. Human ATG4B (Abcam, ab188707) at a dose of 0.5 μg/ml with or without 1 μg/ml of human LCN-2 (R&D Systems, 1757-LC-050) and 5 μM sypro orange (Thermo Fisher, S-6650) were incubated for 30 min at 4° C. The change in Tm was evaluated in a quantitative PCR machine (ABI Systems, USA) using a previously published method (Huynh et al., Curr. Protoc. Protein Sci., 79: 28.9.1-28.9.14 (2015); Layton et al., Protein Sci., 20(8): 1439-50 (2011)).

Single Cell RNA Sequencing (scRNAseq) and Bioinformatics

The sub-retinal region was carefully dissected out from enucleated eyes harvested from 3- and 15-month-old Cryba1^{fl/fl} and Cryba1 cKO mice perfused with saline, to remove peripheral blood from the body. The tissues were then subjected to single cell preparation as previously described. scRNAseq was performed as a paid service from the Genomics Research Core of University of Pittsburgh, to identify the RNA expression profile of different cells, particularly of the RPE. Bioinformatics analysis was performed by creating Seurat objects for each sample by the function “CreateSeuratObject” in Seurat package (min.cells=3, min.features=200), and cells were further filtered out with nFeature_RNA>8000 or with a mitochondrial rate>20%. Scrublet to remove predicted doublets with default parameters was used. As a result, 10455 cells were used for downstream analysis. After clustering the cells, cell type identities were assigned based on the top marker genes of each cluster as well as visualizing the expression of canonical marker genes of candidate cell types. Average expression values of the genes in each cell type and each sample were then calculated by the function “AverageExpression” in Seurat package (Hao et al., Cell, 184(13): 3573-3587.e29 2021)).

### Estimation of Autophagosome Number in RPE Cells

ARPE19 cells were infected for 12 h with 10⁸ vg/ml of an Adenovirus-GFP-RFP-LC3 construct (Vector Biolabs, 2001). The cells were then treated with 10⁷ vg/ml of an Ad-LCN-2 construct (as described above) for 48 h. The cells were fixed with 2% paraformaldehyde (PFA, Alfa Aesar, J61899-AP) for 30 min at 4° C. and then the cells were labeled for F-actin with Alexa 647 phalloidin (Invitrogen, A22287) and nuclei with Hoeschts (Sigma, B2883) 1 mg/100 ml dH₂O. Confocal images were acquired at 60× using Nikon NIS Elements v5.3(Nikon, Melville NY). 3D spot detection was used to segment positive structures for size, shape and intensity for RFP/GFP, and RFP only puncta were identified.

### Western Blot

Whole cells lysates were made from RPE cells harvested from Cryba1^{fl/fl} or Cryba1 cKO mice or from in vitro RPE flat mount cultures from different experimental conditions as explained previously. The lysates were either made in 1×RIPA buffer (EMD Milipore, Cat #20-188) or NATIVE lysis buffer (Abcam, ab156035) containing 0.1% of a protease inhibitor cocktail (Sigma Aldrich, 13786) and 0.1% phosphatase inhibitor cocktail (Sigma Aldrich, P0044-5ML). The protein extracts were subjected to quantification by using the Pierce BCA Protein Assay Kit (Thermo Fisher, 23225). The RIPA protein samples were mixed with 4× protein sample buffer (Life Technologies, NP0007) containing 5% 2-mercaptoethanol (Sigma Aldrich, M6256) and heated at 95° C. for 10 min to denature. The native protein samples were mixed with NATIVE sample buffer (Bio-Rad, 1610738) at a 1:1 ratio. The native samples were loaded on NATIVE-PAGE gels (Thermo Fisher, BN1002BOX) and run using NATIVE-PAGE running buffer (Thermo Fisher, BN2001). Samples were loaded onto a 4-12% Bis-Tris Nu-PAGE gel (Invitrogen, NP0323BOX) and run with MES buffer (Invitrogen, NP0002). Proteins were transferred to nitrocellulose membranes (Invitrogen, 1B23001), which were then blocked in 5% blocking grade skim milk (Bio-Rad, 170-6404) or 5% BSA (Sigma Aldrich, A3912-50G) for phosphorylated proteins. The membranes were incubated with the appropriate primary antibody overnight followed by horseradish peroxidase-conjugated anti-rabbit (KPL, 074-1506) or anti-mouse (KPL, 074-1806) secondary antibodies for 1 h at RT. A chemiluminescence development technique was utilized to develop the blots (GE Healthcare, RPN2209) and the blots were imaged using the Azure imaging system (Azure Biosystems, USA). Densitometry was performed to estimate the protein expression relative to the loading control (ACTIN or VINCULIN) using ImageJ software (National Institute of Health, USA) (Ghosh et al., J Pathol., 241(5): 583-588 (2017); Shang et al., Aging Cell, 16(2): 349-359 (2017)).

### Human Samples

The human RPE donor samples (˜78 years old, both male and female) from AMD patients and age-matched control subjects (with no noticeable ophthalmological abnormalities) were procured from Lions Gift of Sight, Minnesota. The inferior region of the total RPE was lysed in NATIVE lysis buffer (Abcam, ab156035) containing 0.1% protease inhibitor cocktail (Sigma Aldrich, 13786) and 0.1% phosphatase inhibitor cocktail (Sigma Aldrich, P0044-5ML). Western blot was performed as previously described to for LCN-2 using a monoclonal antibody (mLCN-2 Clone #6) (Ghosh et al., J. Pathol., 241(5): 583-588 (2017); Shang et al., Aging Cell, 16(2): 349-359 (2017)).

### Assessment of Autophagy Flux

Autophagy flux in RPE cells was assessed as previously described. Briefly, Cryba1^{fl/fl} RPE explants were infected with either Ad-LCN-2 at a dose of 10⁷ vg/ml for 48 h (medium was changed after 24 h of infection) or left untreated. ChQ (50 PM) was added for the last 6 h of the experimental duration. Cells were lysed in 1×RIPA containing 0.1% each of protease and phosphatase inhibitor cocktails and Western blot was performed to evaluate the levels of LC3-I and LC3-II. Autophagy flux was estimated by calculating the ratio of LC3-II in chloroquine (ChQ)-treated cells with respect to untreated cells (Valapala et al., Autophagy, 10(3): 480-96 (2014); Yazdankhah et al., Autophagy, 17(10): 3140-3159 (2021)).

### Cell Free ATG4B Activity Assay

ATG4B activity assay was measured by incubating recombinant human LC3-GST (Enzo, BML-UW1155-0500) at a dose of 20 μg/ml with 0.5 μg/ml of human ATG4B (Abcam, ab188707) with or without human LCN-2 (R&D Systems, 1757-LC-050) at a dose of 1 μg/ml for 15 min at room temperature (Pengo et al., Nat. Commun., 8(1): 294 (2017)). The protein samples were then mixed with 4× protein sample buffer (Life Technologies, NP0007) containing 5% 2-mercaptoethanol (Sigma Aldrich, M6256) and heated at 95° C. for 10 min to denature. The samples were then subjected to Western blot using the anti-GST primary antibody (Cell Signaling USA, 2622S) as previously explained (Ghosh et al., J. Pathol., 241(5): 583-588 (2017); Shang et al., Aging Cell, 16(2): 349-359 (2017)).

### Enzyme-Linked Immunosorbent Assay (ELISA)

ELISA was performed using 10-month-old Cryba1^{fl/fl} and cKO RPE lysates as explained previously. Briefly, the RPE choroid complexes were homogenized in 300 μL of complete extraction buffer (Abcam, ab193970). This was followed by performing the ELISA in 96-well microtiter plates (Sigma Aldrich, M9410-1CS) coated with tissue lysates and incubated overnight at 4° C. The plates were then blocked with 5% BSA for 2 h and after washing with PBS, 50 μl of IL-1β antibody (Abcam, ab9722) diluted to 1:1000 was added to each well and incubated for 2 h at RT. Bound cytokines were detected with secondary IgG-HRP (KPL, 074-1506). The color was developed with TMB substrate solution (Thermo Fisher, 34021). The reaction was stopped with 2 N H₂SO₄ solution and absorbance was measured at 450 nm using a microplate reader (Ghosh et al., Commun. Biol., 2: 348 (2019)).

### Intracellular Iron Levels

The intracellular iron levels in RPE cells from in vivo and in vitro experiments were evaluated by following the manufacturer's protocol of the Iron Assay kit (Abcam, ab83366).

### Intracellular Reactive Oxygen Species

The ROS generation in 10-month-old RPE cells from Cryba1^{fl/fl} and Cryba1 cKO mice was evaluated as described previously (Ghosh et al., Cell Signal., 68: 109521 (2020)). Briefly, a 10% tissue homogenate was prepared in PBS and centrifuged at 1800×g for 10 mn, followed by centrifugation at 31,500×g for 10 min to obtain the pellet. The obtained pellet was dissolved in HEPES buffer, pH 7.4 (Sigma Aldrich, H7006-100G). The sample was incubated for 15 min at 37° C. with 5 μM dihydroethidium (DHE; Fisher Scientific, D11347). Fluorescent signals were recorded from the conversion of DHE to 2-hydroxyethidium at the end of the incubation period, at an excitation wavelength of 480 nm and an emission wavelength of 525 nm in a spectrofluorometer (Jasco, USA). The ROS levels in the samples were estimated against a standard curve for an increasing concentration of DHE and were represented as nmoles of DHE formed/mg of protein (Ghosh et al., Cell Signal., 68: 109521 (2020)).

### Live Cell Imaging

To assess the levels of lipid peroxidation in RPE cells, ARPE19 cells were cultured on four chambered dishes (VWR, 627871) and then either left untreated (control) or treated with 250 μM of ferric ammonium citrate (FAC) for 72 h to mimic the increased iron levels in vivo, followed by Ad-LCN-2 (10⁷ vg/ml) for the last 48 h and chloroquine (50 μM) for the last 6 h of the experimental duration respectively. RSL3 (3 μM, overnight; Cayman Chemicals, 19288) was used as a positive control for ferroptosis activation. The clone #6 monoclonal antibody targeting LCN-2 (mAb; 1 μg/ml, 24 h) antibody and 25 μM (overnight) of ferrostatin-1 (Cayman Chemicals, 17729) were added to the culture to rescue ferroptosis induction in the RPE cells. Prior to imaging, the cells were incubated with Liperfluo (10 μM, Fisher Scientific, L24810) for 30 min, and washed twice with PBS before replacing the original media in each chamber. The dishes were then inserted into a closed, thermo-controlled (37° C.) stage top incubator (Tokai Hit Co.) above the motorized stage of an inverted Nikon TiE fluorescent microscope equipped with a 20×optic (Nikon, CFI Plan Fluor, NA 0.75). Liperfluo was excited using a diode-pumped light engine (SPECTRA X, Lumencor) and detected using an Prime95b sCMOS camera (Photometrics) and excitation and emission filters from Chroma. Data were collected every 15 min for 4 h, on approximately 250-500 cells per stage position, by acquiring a large area stitched montage image of each of the 4 separate experimental conditions. Data was collected and liperfluo emissions were analyzed using NIS Elements (Nikon, Inc. Melville, NY).

Transfection with GFP-LC3-RFP-ΔLC3 Construct and Flowcytometry

ARPE19 cells were cultured up to 60% confluency and then transfected with the GFP-LC3-RFP-ΔLC3 construct (Addgene, 84572), to assess ATG4B activity, using the manufacturer's protocol of the Lipofectamine 3000 Transfection Reagent (Thermo Fisher, L3000008) for 72 h (Ghosh et al., Commun. Biol., 4(1): 248 (2021)). The transfection efficiency was confirmed by visualizing the GFP and RFP signals in the transfected cells using a fluorescence microscope. This was followed by infection with an Ad-LCN-2 construct (as described above) for 48 h and treatment with recombinant human LCN-2 protein (R&D Systems, 1757-LC-050) at a dose of 1 μg/ml for 24 h in separate experiments. The cells were then scraped off the plates and were analyzed by flow cytometry using the LSR-II platform (Beckman Coulter, USA). The results were analyzed using the FlowJo software (v10.8) and the data from each group was represented as the ratio of median GFP fluorescence to RFP fluorescence (Kaizuka et al., Mol. Cell, 64(4): 835-849 (2016); Ghosh et al., Commun. Biol., 4(1): 248 (2021)).

### Glutathione Peroxidase Activity and Malondialdehyde Levels

RPE cell lysates from in vivo experimental groups were subjected to assessment of GPX4 activity and MDA levels, markers of ferroptosis activation, by following the manufacturer's protocol of the Glutathione Peroxidase Assay Kit (Abcam, ab102530) and Lipid Peroxidation Assay Kit (Thermo Fisher, MAK085-1KT), respectively.

### LCN-2 Knockdown in RPE Explants

Lcn2 shRNA lentiviral (Santa Cruz Biotechnology, sc-60044-V) and control shRNA (Santa Cruz Biotechnology, sc-108080) were used at a concentration of 10⁸ vg/ml to infect 10-month-old Cryba1 KO RPE explants for 72 h as previously described (Ghosh et al., Commun. Biol., 2: 348 (2019)). Western blot was performed to assess the levels of the LC3-II/LC3-I ratio in these cells relative to control, as previously explained (Ghosh et al., J Pathol., 241(5): 583-588 (2017); Shang et al., Aging Cell, 16(2): 349-359 (2017)).

### Computer Modelling of Protein Binding

The crystal structure of human ATG4B-LC3 (1-120) complex (PDB: 2z0d.pdb) and LCN-2 homodimer (PDB: 1qqs.pdb) were obtained from the RCSB PDB database (rcsb.org/). The possible interaction between LCN-2 or ATG4B and LC3 was tested using an interactive protein docking and molecular superposition program HEX protein docking program, version 6.3 (//hex.loria.fr/dist63/) as explained previously. Models of proteins were visualized using UCSF Chimera (cgl.ucsf.edu/chimera).

### Statistical Analysis

All analyses were performed using the GraphPad 8.0 software was used to perform statistical analysis. Students' t-test and one-way ANOVA followed by Tukey's post hoc test to measure the differences between groups (for experiments involving more than two groups) was performed. The significance was set at P<0.05 and all p-values less than 0.05 (*P<0.05) and 0.01 (**P<0.01) were considered significant. The analyses were performed on triplicate technical replicates. Results are presented as mean±standard deviation (SD) (Ghosh et al., Commun. Biol., 2: 348 (2019); Ghosh et al., J. Pathol., 241(5): 583-588 (2017); Shang et al., Aging Cell, 16(2): 349-359 (2017); Ghosh et al., Commun. Biol., 4(1): 248 (2021)).

### Abbreviations

The following abbreviations are employed in the descriptions of the experiments described in Examples 1-9: Ad-GFP: Adenovirus-Green Fluorescent Protein; Ad-LCN-2-GFP: Adenovirus-LCN-2-Green Fluorescent Protein; Ad-LCN-2: Adenovirus-Lipocalin-2; Akt2: AKT Serine/Threonine Kinase 2; AMBRA1: Activating molecule in BECN1-regulated autophagy protein 1; AMD: Age-related macular degeneration; ARPE19: Adult Retinal Pigment Epithelial cell line-19; Asp 278: Aspartate 278; ATG4B: Autophagy Related 4B Cysteine Peptidase; ATG4C: Autophagy Related 4C Cysteine Peptidase; ATG7: Autophagy Related Protein 7; ATG9B: Autophagy Related Protein 9B; BLOC-1: Biogenesis of lysosome-related organelle complex1; BLOC1S1: Biogenesis of lysosomal organelles complex 1 subunit 1; C57BL/6J: C57 black 6J; cGAS: Cyclic GMP-AMP synthase; ChQ: Chloroquine; cKO: Conditional knockout; Cys 74: Cysteine 74; Dab2: DAB Adaptor Protein 2; Def: Deferoxamine; DHE: Dihydroethidium; DMSO: Dimethyl Sulfoxide; ERG: Electroretinography; FAC: Ferric ammonium citrate; Fe⁺²: Ferrous; FTH1: Ferritin Heavy Chain 1; GST: Glutathione S-transferase; GT: Golden ticket; H2O2: Hydrogen peroxide; His 280: Histidine 280; IFNλ: Interferon lambda; IL-1β: Interleukin 1 beta; Integrin β1: Integrin beta 1; IS: Inner segment; KO: Knockout; LC3-GST: Microtubule-associated protein 1A/1B-light chain 3-Glutathione S-transferase; LC3-I: Microtubule-associated protein 1A/1B-light chain 3-I; LC3-II: Microtubule-associated protein 1A/1B-light chain 3-II; LC3A: Microtubule-associated proteins 1A/1B light chain 3A; LC3B: Microtubule-associated proteins 1A/1B light chain 3B; LCN-2: Lipocalin-2; mAb: Monoclonal antibody; MDA: malondialdehyde; NLRP3: NLR family pyrin domain containing 3; NOD-SCID: Nonobese diabetic-severe combined immunodeficiency; OS: Outer Segment; P62/SQSTM1: Ubiquitin-binding protein p62/Sequestosome 1; PBS: Phosphate buffered saline; RFP: Red Fluorescent Protein; rLCN-2: recombinant lipocalin-2; ROS: Reactive oxygen species; RPE SM: Retinal pigmented epithelium spent medium; RPE: Retinal pigmented epithelium; RSL3: RAS-selective lethal; scRNAseq: single cell ribonucleic acid sequencing; SD-OCT: Spectral domain optical coherence tomography; shRNA: Small hairpin ribonucleic acid; SM: Spent medium; SOD-1: superoxide dismutase-1; STAT1: Signal transducer and activator of transcription 1; STING: Stimulator of interferon genes; WT: Wild type.

### Example 1

This example demonstrates that LCN-2 binds to ATG4B and modulates autophagy in the RPE.

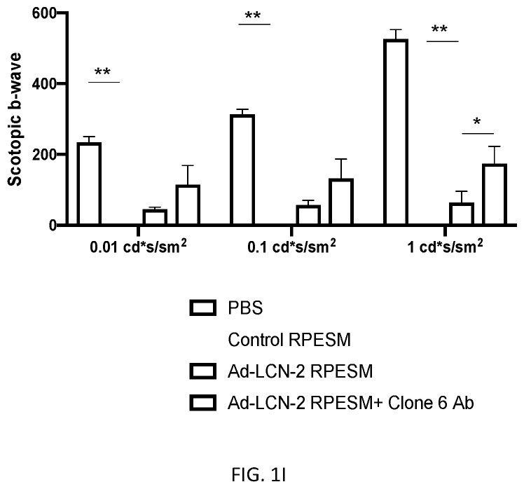

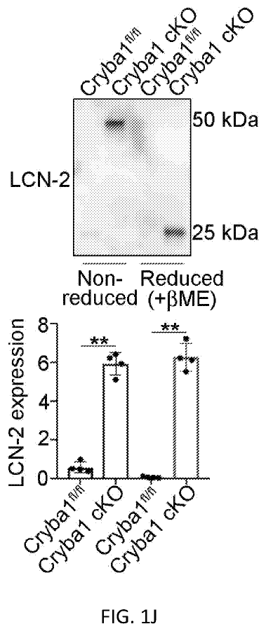

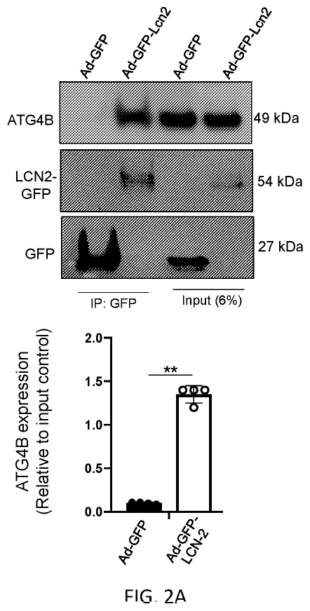

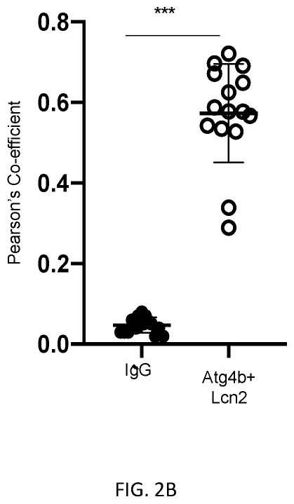

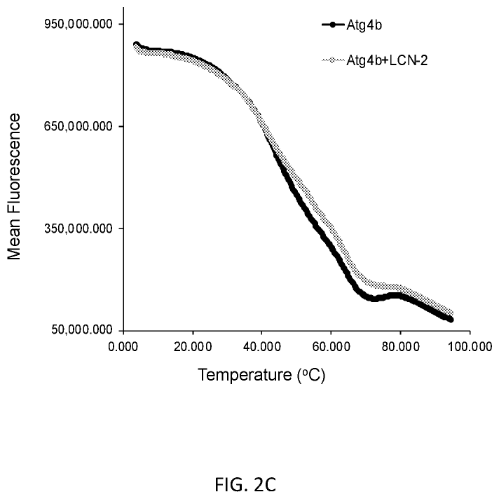

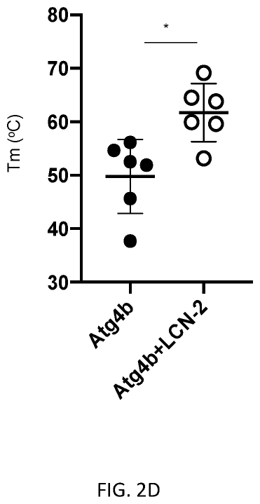

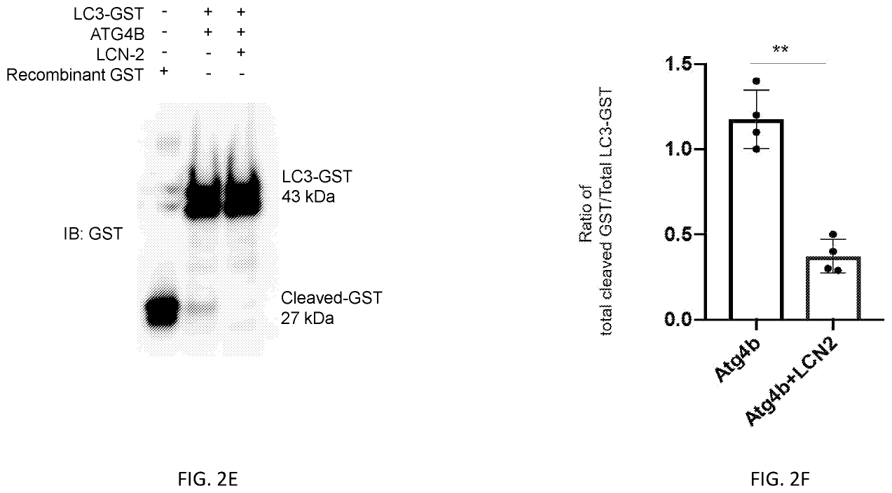

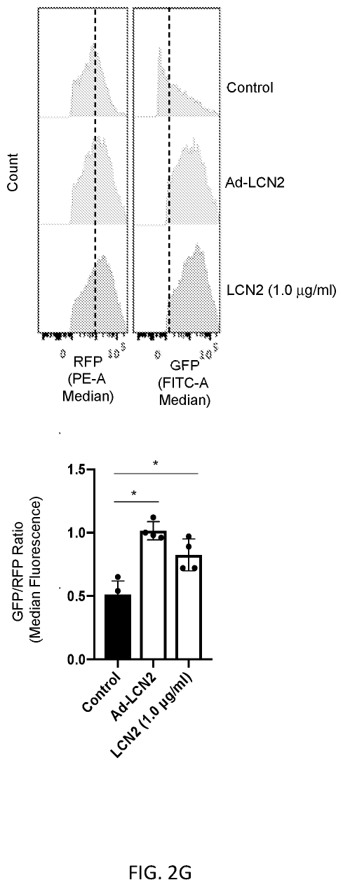

Elevated LCN-2 can deregulate autophagy in several cell types (Chan et al., Mol. Cell Endocrinol., 430: 68-76 (2016); Sung et al., J Cell Physiol., 232(8): 2125-2134 (2017)). In a human proteome high-throughput array on RPE cells, it was found that LCN-2 interacts with ATG4B, a cysteine protease that regulates LC3B processing (lipidation and delipidation) during autophagy, a process involved in cellular homeostasis (Ghosh et al., Commun. Biol., 2: 348 (2019); Maruyama et al., J. Antibiot. (Tokyo), 71(1): 72-8 (2017); Dikic et al., Nat. Rev. Mol. Cell Biol., 19(6): 349-364 (2018)). The binding between LCN-2 and ATG4B in RPE cells was shown by overexpressing Adenovirus-LCN-2-Green Fluorescent Protein (Ad-LCN-2-GFP) and Adenovirus-GFP (Ad-GFP, vector control) constructs in cultured C57BL6/J mouse RPE explants. In a pull-down assay with anti-GFP magnetic beads, it was confirmed that ATG4B binds to LCN-2 in the lysates of Ad-LCN-2-GFP infected cells, but not in Ad-GFP infected cells (FIG. 2A). Furthermore, ARPE19 cells infected with Ad-LCN-2-GFP and subsequently immunostained with an ATG4B antibody showed a similar association of the proteins (FIG. 2B). In addition, a thermal shift assay was performed with sypro orange dye, which assesses the stability of a protein-protein complex upon heat denaturation, thereby indicating the binding ability/strength of the complex (Huynh et al., Curr. Protoc. Protein Sci., 79: 28.9.1-28.9.14 (2015); Layton et al., Protein Sci., 20(8): 1439-50 (2011)). The melting temperature (Tm) shift in human recombinant ATG4B upon incubation with human recombinant LCN-2 was consistent with binding between the two proteins (FIGS. 2C-2D). A complex involving ATG4B, LCN-2, and LC3 was also predicted by molecular modeling. LCN-2 can interact with the catalytic site of ATG4B, including residues Cysteine 74 (Cys 74), Aspartate 278 (Asp 278), and Histidine 280 (His 280), where LC3 is cleaved. The pull-down assays and molecular docking suggest that LCN-2 binds to the ATG4B catalytic site. Therefore, it is highly likely that LCN-2 can modulate the C-terminal end cleavage of pro-LC3 to generate LC3-I, which is subsequently lipidated to generate active LC3-II.

Since molecular docking suggested that LCN-2 binds at the ATG4B catalytic site, a cell-free ATG4B activity assay was performed using recombinant human LC3-Glutathione S-transferase (LC3-GST) protein. LC3-GST was incubated with recombinant human ATG4B (0.5 μg/ml), and the efficacy of GST-cleavage in the presence or absence of human recombinant LCN-2 (1.0 μg/ml, 15 minutes, room temperature), was quantified by Western blotting with anti-GST antibody. Decreased GST cleavage at the C-terminal end of LC3 was observed in the presence of LCN-2 compared to controls (FIG. 2E-2F). Further, ARPE19 cells were transfected with a GFP-LC3-RFP-ΔLC3 fluorescent construct, a probe that specifically evaluates ATG4B-mediated LC3 processing (ATG4B activity) and autophagy flux in cells by evaluating the GFP:RFP signal as previously described (Kaizuka et al., Mol. Cell, 64(4): 835-849 (2016)). Post transfection, when cells were either untreated, treated with recombinant LCN-2 (1.0 μg/ml) for 24 h as a positive control, or infected with an Ad-LCN-2 construct for 48 h and then analyzed by flow cytometry, GFP fluorescence was increased in the recombinant LCN-2 and Ad-LCN-2 treated cells (FIG. 2G), indicating impaired LC3 processing/lipidation.

### Example 2

This example demonstrates that increased LCN-2 attenuates autophagy in RPE cells.

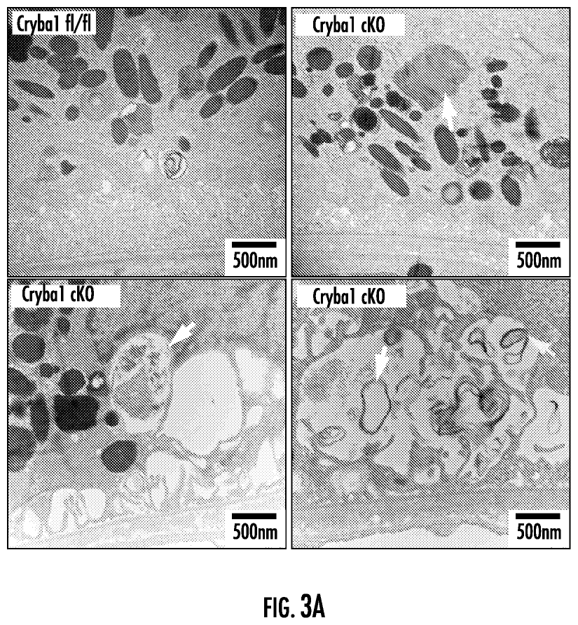

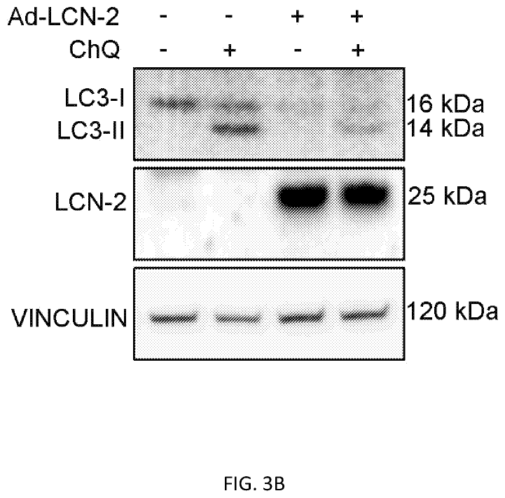

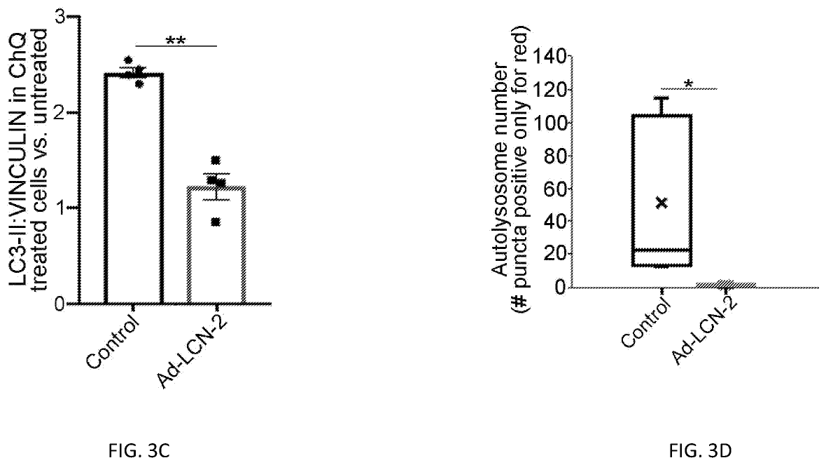

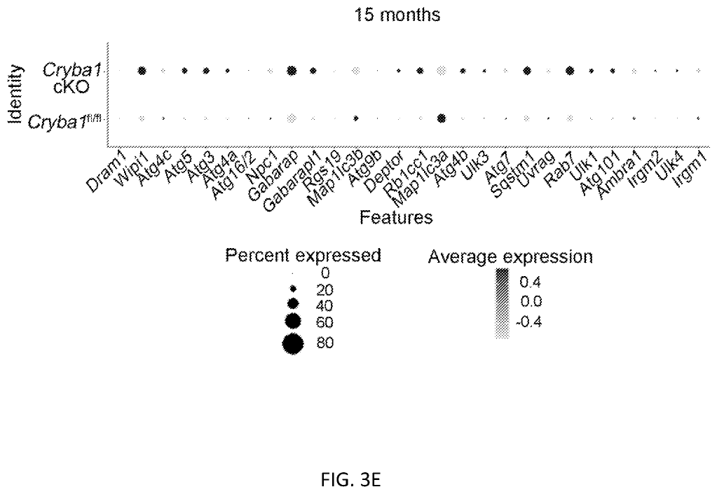

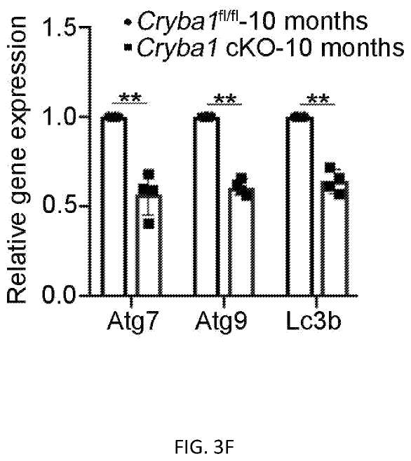

To further evaluate the functional connection between LCN-2 and autophagy in the RPE, the underlying signaling cascades were investigated in a genetically engineered animal model which lacks Cryba1 (encodes for PA3/A1-crystallin, an endolysosomal protein) specifically in the RPE (conditional knockout; cKO) (Ghosh et al., Commun. Biol., 2: 348 (2019); Valapala et al., Aging Cell, 13: 1091-4 (2014); Valapala et al., Autophagy, 10: 480-496 (2014)). Loss of Cryba1 decreases lysosomal function by activating mTORC1 signaling decreasing autophagy (Valapala et al., Autophagy, 10: 480-496 (2014); Shang et al., Aging Cell, 16(2): 349-359 (2017)). The Cryba1 cKO is a well-characterized age-dependent mouse model with a dry-AMD like phenotype (Ghosh et al., Commun. Biol., 2: 348 (2019); Valapala et al., Aging Cell, 13: 1091-4 (2014); Valapala et al., Autophagy, 10: 480-496 (2014)). In the RPE cells of the Cryba1 cKO mouse, as well as in human AMD donor samples, autophagy is inhibited (Intartaglia et al., FEBS J. (2021); Valapala et al., Autophagy, 10: 480-496 (2014)). Increased accumulation of undigested photoreceptor outer segments (arrow; FIG. 3A, upper right) and double membrane autophagosomes in aged (20 months old) Cryba1 cKO mice (arrows; FIG. 3A, bottom left and bottom right) was shown, relative to age-matched floxed controls (FIG. 3A, upper left). It is highly likely that abnormal accumulation of autophagosomes, which have not fused with lysosomes, results from alterations in autophagosome processing. Therefore, it was investigated whether autophagosome processing in the RPE of aged Cryba1 cKO mice is compromised by increased LCN-2. Single cell RNA sequencing (scRNAseq) analysis of cells in the sub-retinal space (which includes the RPE) between the photoreceptors and the choroid in 15-month-old Cryba1-floxed and cKO mice was performed when LCN-2 was upregulated in the RPE cells of the cKO animals (Valapala et al., Aging Cell, 13: 1091-4 (2014). It is now well established that the dynamic interactions between different immune cells and the RPE cells in the sub-retinal space contributes to AMD pathogenesis (Tan et al., Int. J Biol. Sci., 16(15): 2989-3001 (2020); Yu et al., Trends Neurosci., 43(6): 433-449 (2020). While the scRNAseq analysis identified several cell clusters, autophagy-specific gene expression in the RPE cell cluster by identifying the expression of RPE-specific markers like Rpe65 and Rlbp1 was the focus of further study. AMBRA1, ATG4C, ATG9B, ATG7 as well as LC3A&B were downregulated in the RPE of Cryba1 cKO mice (FIG. 3E), and several other autophagy-related genes were noticeably differentially expressed (FIG. 3E). These results were confirmed by quantitative PCR analysis (FIG. 3F), suggesting that LCN-2 might also play a pivotal role in the autophagy process of Cryba1 cKO.

Also explored was whether LCN-2 in RPE cells could alter autophagy flux because it modulates ATG4B activity (FIG. 12). WT RPE explants in culture were infected with Ad-LCN-2 for 48 h or left untreated, with or without addition of chloroquine (ChQ) (Mauthe et al., Autophagy, 14(8): 1435-1455 (2018)), a lysosomotropic agent known to inhibit autophagy for the last 6 h of the experiment. As seen in the histogram in FIG. 3C, LC3-II flux in the Ad-LCN-2 treated explants was significantly decreased relative to untreated controls (FIGS. 3B-3C). To determine if LCN-2 specifically affects autophagosome formation, human ARPE19 cells were infected with the Ad-LCN-2 construct or left untreated (control). An overnight infection with an Adenovirus-GFP-RFP-LC3B construct was performed to label the autophagosomes yellow and autolysosomes red. The number of red puncta (autolysosomes) was significantly decreased in Ad-LCN-2 infected cells when compared with controls (FIG. 3D), consistent with decreased autophagy flux in RPE cells overexpressing LCN-2. To further provide evidence that LCN-2 can modulate autophagy flux, RPE explants from 10-month-old Cryba1 KO mice, which show elevated LCN-2, were cultured and infected with either lentiviral-LCN-2-shRNA or lentiviral control-shRNA particles for 72 hours. Assessment of LC3-TT levels by Western blot showed that LCN-2 knockdown significantly restored the autophagy flux in Cryba1 KO RPE cells in vitro compared to control-shRNA treated cells.

### Example 3

This example demonstrates that alterations in autophagosome processing and iron regulation in the RPE trigger inflammasome activation through the cGAS/STING pathway.

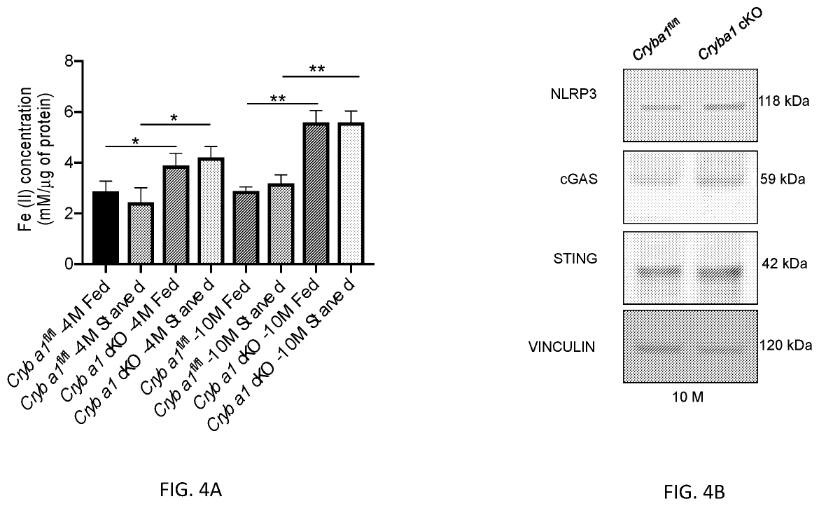

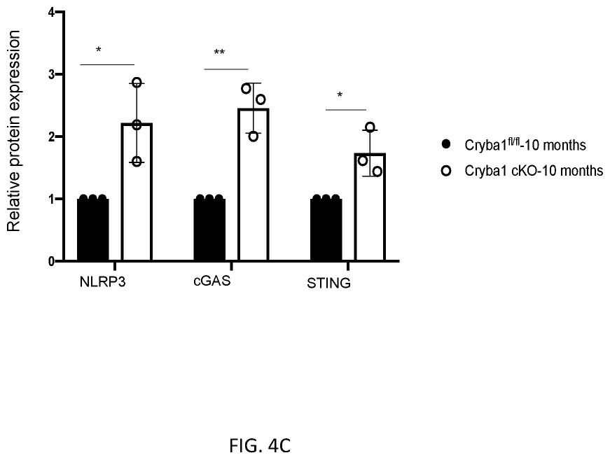

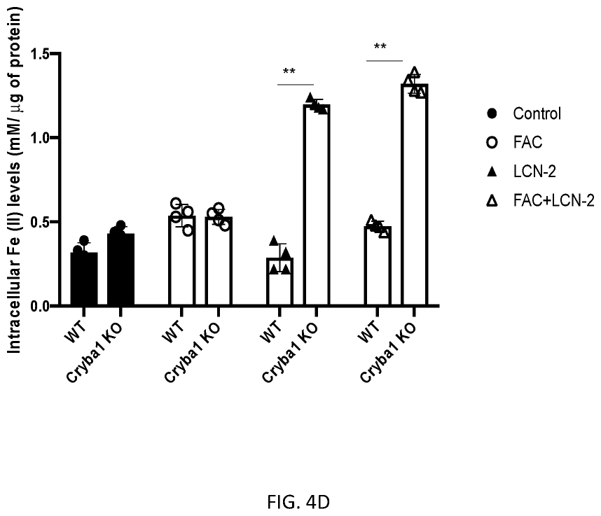

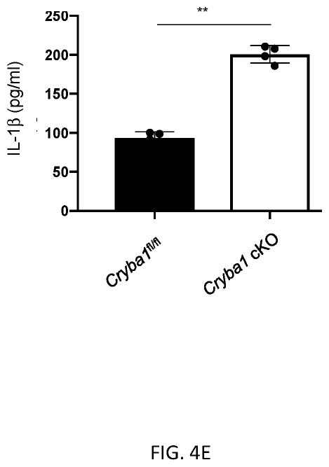

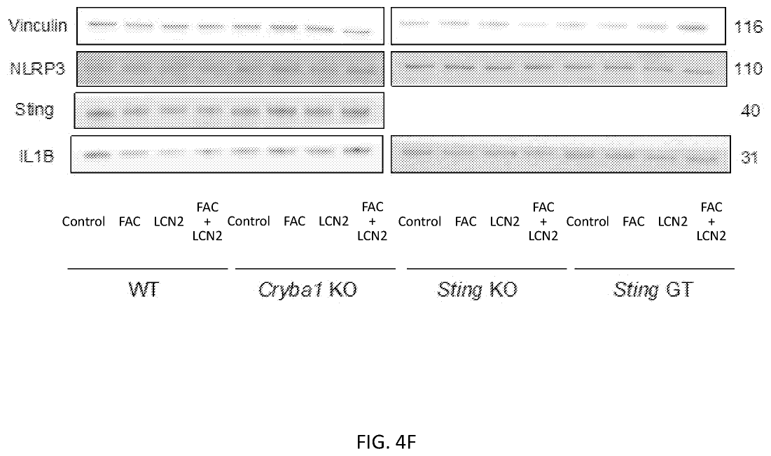

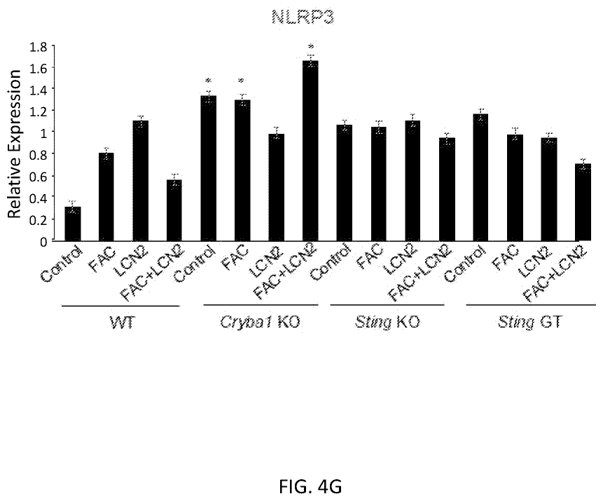

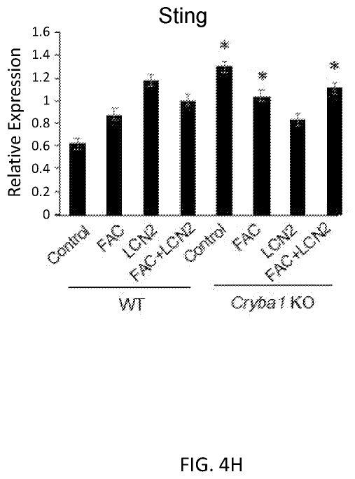

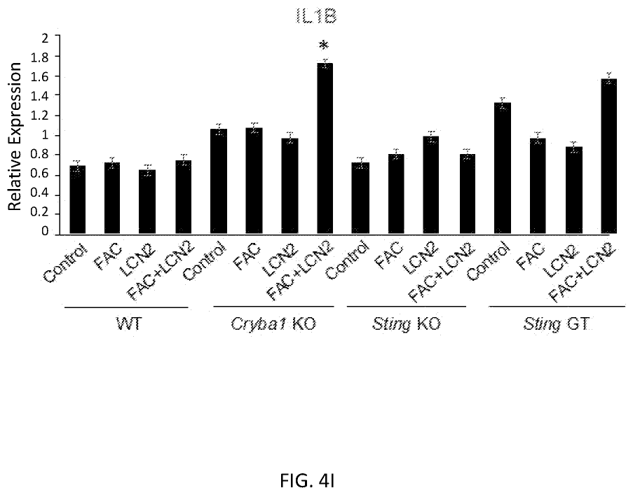

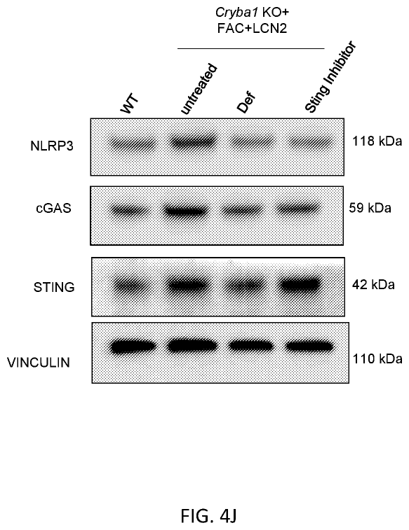

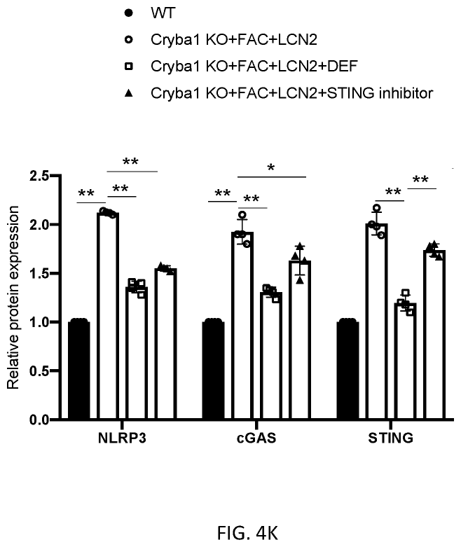

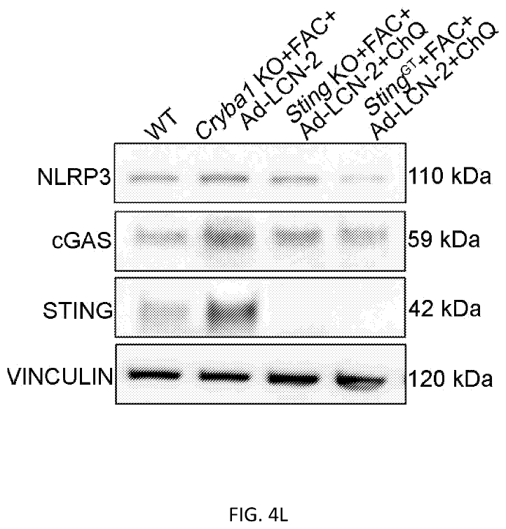

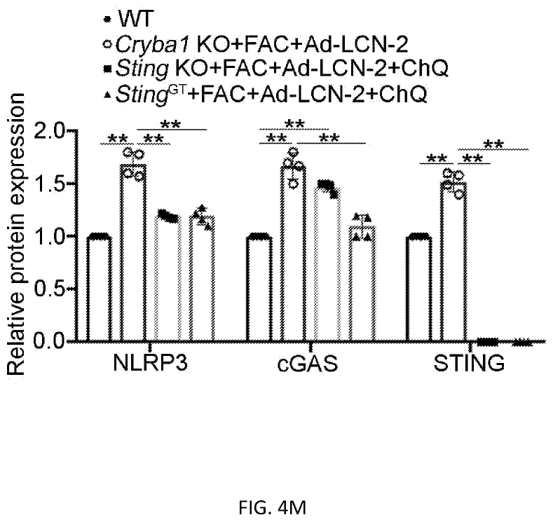

The effect of impaired autophagy on iron accumulation and the consequent iron-induced inflammasome activation is well-documented in several diseases, including AMD (Jacomin et al., Front. Cell Dev. Biol., 7: 142 (2019); Masaldan et al., Redox Biol. 14: 100-115 (2018); Hedbrant et al., Mediators Inflamm. 2020: 8490908 (2020); Li et al., Dev Cell. 46(4): 441-455.e8 (2018); Gelfand et al., Cell Rep. 11(11): 1686-93 (2015); Handa et al., Nat Commun., 10(1): 3347 (2019)). Inflammasome mediators are cleared by autophagy, thereby promoting resolution of inflammation in cells (Biasizzo et al., Front Immunol. 11: 591803 (2020)); conversely, their accumulation has been reported to be associated with the progression of several diseases (Zhao et al., Front Cell Dev. Biol., 9: 657478 (2021)). Moreover, LCN-2 is a potent iron chelator that is sequestered in lysosomes (Halaas et al., J. Infect Dis., 201(5): 783-92 (2010)). It was speculated that increased LCN-2 and dysfunctional lysosomes in the aged Cryba1 cKO RPE along with consequent decrease in autophagy might alter iron homeostasis and trigger iron-mediated inflammasome activation in RPE cells. Consistent with this premise, it was found that redox sensitive iron (ferrous ion; Fe²⁺) levels were elevated in Cryba1 cKO RPE cells, compared to age-matched controls (FIG. 4A). Autophagy induction in vivo following fasting did not produce any noticeable decrease in iron accumulation in the Cryba1 cKO RPE (FIG. 4A). To determine if functional lysosomes are required for LCN-2 dependent iron homeostasis, RPE explants were harvested from 4 month-old mice (WT and Cryba1 complete knockout; KO). This approach has been used in the in vitro experiments since in Cryba1 KO mice, PA3/A1-crystallin is completely absent in all RPE cells, but only 85% of the RPE cells lack PA3/A1-crystallin in the cKO because of the mosaic expression of Best1 (Best1-Cre mice were used to generate Cryba1 cKO) (Iacovelli et al., Invest. Ophthalmol. Vis. Sci., 52(3): 1378-83 (2011); Valapala et al., Autophagy, 10: 480-496 (2014)). The explants were cultured were cultured with or without ferric ammonium citrate (FAC; 250 μM) for 72 h, followed by infection with Ad-LCN-2 for 48 h. Ferrous ion levels were significantly higher in Cryba1 KO RPE cells even in the presence of the iron chelator, LCN-2 (FIG. 4D). However, WT cells treated with FAC and LCN-2 did not show any noticeable increase in intracellular ferrous iron compared to untreated controls (FIG. 4D). When WT cells were treated with ChQ, for 6 h following FAC and Ad-LCN-2 treatment, ferrous ion accumulated. It was concluded that normal lysosomal function is needed for LCN-2-dependent iron regulation in RPE cells.

Previous studies have elegantly shown that iron accumulation in the RPE activates the inflammasome in AMD (Gelfand et al., Cell Rep. 11(11): 1686-93 (2015); Handa et al., Nat Commun., 10(1): 3347 (2019)). Since ferrous ion levels are elevated in aged Cryba1 cKO RPE, inflammasome activation was examined. Increased levels of cGAS, STING, and NLRP3 protein, along with increased cleavage of pro-IL-1β into active IL-1β, an indicator of inflammasome activity (Martin-Sinchez et al., Cell Death Differ., 23(7): 1219-31 (2016)), relative to age-matched floxed controls (FIGS. 4B-4C and 4E) were found. To provide further evidence that accumulated iron in RPE cells with impaired lysosomes activates inflammasomes, RPE explants from 4-month-old mice (WT and Cryba1 KO), which show no increase in either LCN-2 or the inflammasome at this age, were cultured. The explants were treated with FAC for 72 h and infected with an Ad-LCN-2 construct for the last 48 h of the experiment. Increased levels of NLRP3, IL-1, and STING protein were found in FAC+LCN-2 treated RPE cells from the Cryba1 KO, but not in WT cells (FIGS. 4F-4H), indicating that functional lysosomes are involved in maintaining cellular iron homeostasis.

Inflammasome activation through the cGAS/STING pathway is observed in several neurodegenerative diseases, including AMD (Decout et al., Nat. Rev. Immunol., 21(9): 548-569 (2021); Kerur et al., Nat. Med., 24(1): 50-61 (2018)). To test if iron-induced inflammasome activation in RPE cells is driven by cGAS/STING pathway, 4 month-old RPE explants from WT, Sting KO and Sting^{Gt} mutant (a mutation in exon 6 of the Sting gene) mice were cultured. The explants were either untreated or treated with FAC for 72 h and Ad-LCN-2 (for the last 48 h) and then ChQ was added for the last 6 hours. FAC, LCN-2 and ChQ treatment to WT, Sting KO, Sting^{Gt} mutants induced iron accumulation. However, the treatment failed to activate the NLRP3 inflammasome (FIGS. 4F-4H) and IL-13 (FIG. 41) secretion in Sting KO or Sting^{Gt} mutant RPE cells due to the lack of functional Sting (FIGS. 4F-4H). In addition, it was also found that LCN-2 overexpression in Sting KO or Sting^{Gt} mutant RPE explants in culture could reduce autophagy flux. These results indicate that even though LCN-2 decreases autophagy flux in cells and iron accumulates with the loss of lysosomal function, the lack of a functional Sting gene prevents inflammasome activation in these cells.

To further prove that the iron accumulation triggers inflammasome activation via the cGAS/STING pathway, Cryba1 KO RPE explant cultures from 4-month-old mice were pre-treated with or without the chelator deferoxamine (Def; 100 M for 24 h (Cappellini et al., Hemoglobin, 33 Suppl 1: 558-69 (2009)), a specific inhibitor of STING (H-151; 1 M for 6 hours (Hong et al., PNAS, 118(24): e2105465118 (2021)), or vehicle control (DMSO). The cultures were then exposed to FAC and Ad-LCN-2. The chelation of intracellular iron with Def or STING inhibition significantly reduced the levels of NLRP3. While both STING and cGAS levels were reduced upon Def treatment in Cryba1 KO RPE cells cultured in the presence of FAC and Ad-LCN-2, such changes were not observed in KO RPE cells treated with the STING inhibitor (FIGS. 4J-4K). Collectively, these results provide evidence that altered lysosomal function and deregulated LCN-2-dependent iron homeostasis activates inflammasome via the cGAS/STING pathway in RPE cells.

### Example 4

This example demonstrates that deregulated LCN-2/iron axis triggers oxidative damage and lipid peroxidation in RPE cells.

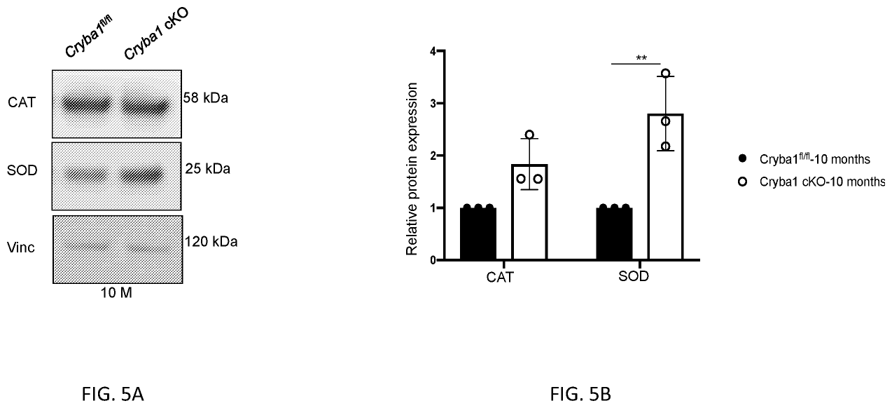

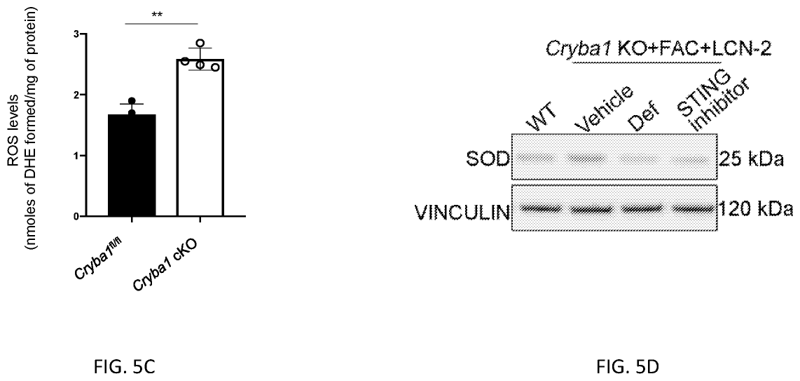

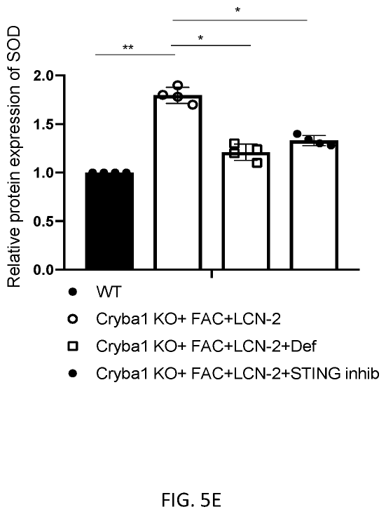

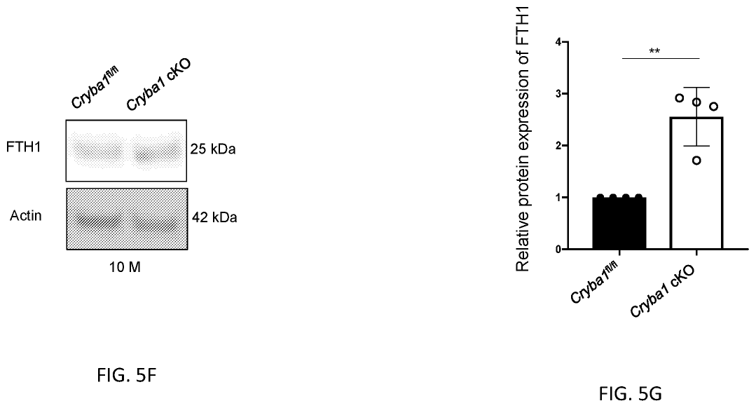

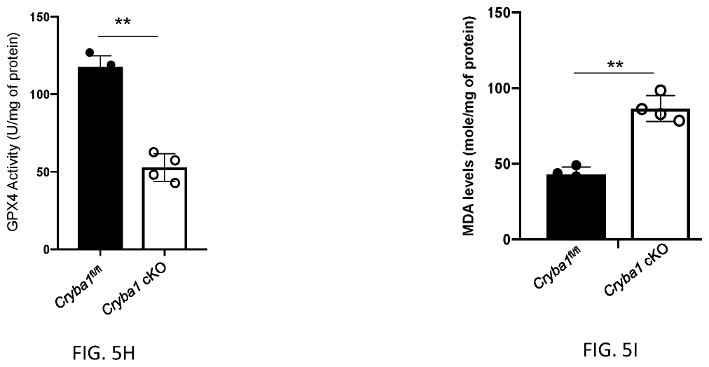

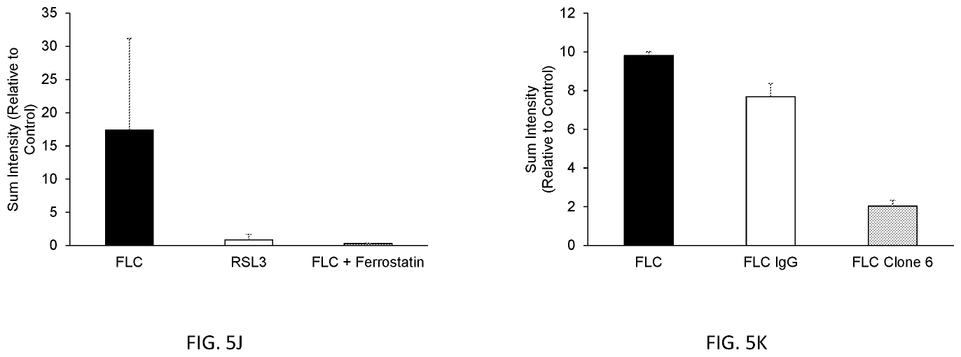

To ascertain if elevated iron and inflammasome activation can cause oxidative damage in the mouse model with an AMD-like phenotype (Valapala et al., Aging Cell, 13: 1091-4 (2014); Valapala et al., Autophagy, 10: 480-496 (2014); Ghosh et al., Commun. Biol., 2: 348 (2019)) the levels of redox regulators in RPE cells from aged (10 month-old) Cryba1 cKO mice were evaluated. Elevated levels of superoxide dismutase-1 (SOD1) in RPE cells from the cKO mice relative to controls (FIG. 5A-5B) were found, which was accompanied by elevated superoxide anion, as visualized by dihydroethidium (DHE) fluorescence (FIG. 5C). These results indicate that SOD1, which neutralizes superoxide anion, is upregulated while catalase, a regulator of H₂O₂ (He et al., Cell Physiol. Biochem., 44(2): 532-553 (2017)), did not show any noticeable change in the aged Cryba1 cKO RPE. Thus, these data indicate that the process of redox changes might be driven by superoxide and not H₂O₂. Moreover, to further establish that iron-induced inflammasome activation is responsible for the oxidative damage in RPE cells, Cryba1 KO RPE explant cultures from 4-month-old mice were pre-treated with Def, STING inhibitor or vehicle control (DMSO), and then were exposed to FAC and Ad-LCN-2. It was found that treatment with Def and the STING inhibitor decreased SOD1 (FIGS. 5D-5E) and ROS levels in explants treated with FAC and Ad-LCN-2, suggesting that iron accumulation and inflammasome activation drive the redox imbalance in these cells. Cellular iron accumulation and subsequent oxidative damage can induce lipid peroxidation and subsequent ferroptosis (Mazhar et al., Cell Death Discov., 7(1): 149 (2021); Li et al., Front. Cell Dev. Biol., 9: 698679 (2021)). However, the role of lipid peroxidation and ferroptosis in AMD is unclear. To address the hypothesis that the abnormal iron-mediated inflammasome activation and oxidative stress in the RPE triggers lipid peroxidation or ferroptosis, the levels of ferroptosis and lipid peroxidation markers, such as ferritin heavy chain 1 (FTH1) and malondialdehyde (MDA) (Li et al., Cell Death Dis., 11(2): 88 (2021)), along with the activity of the antioxidant enzyme glutathione peroxidase (He et al., Cell Physiol. Biochem., 44(2): 532-553 (2017)), was quantified in RPE cells from Cryba1 cKO mice. It was found that FTH1 protein expression was highly elevated in 10-month-old Cryba1 cKO RPE (FIGS. 5F-5G) and that MDA was increased (FIG. 5I) while glutathione peroxidase activity was decreased (FIG. 5H) in the same animals, compared to controls.

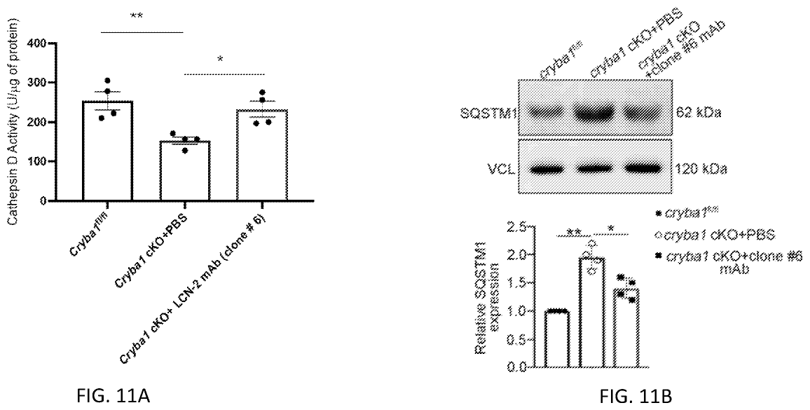

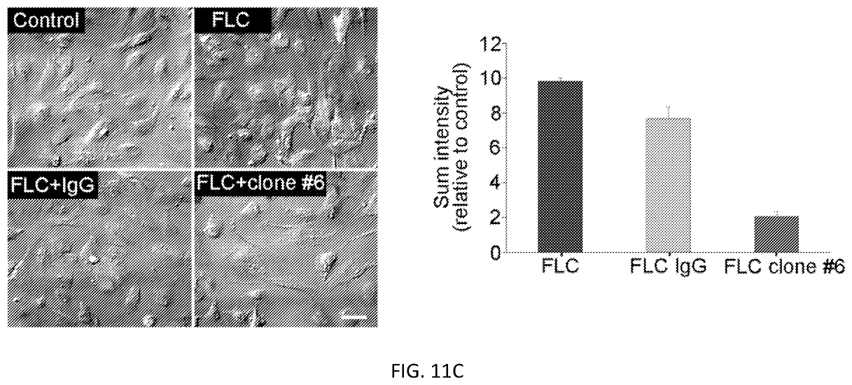

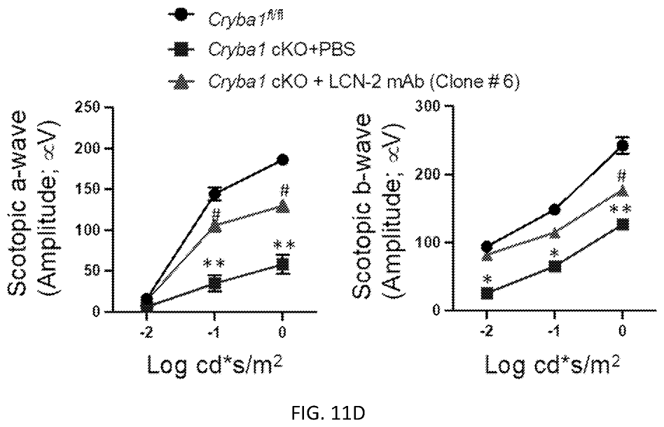

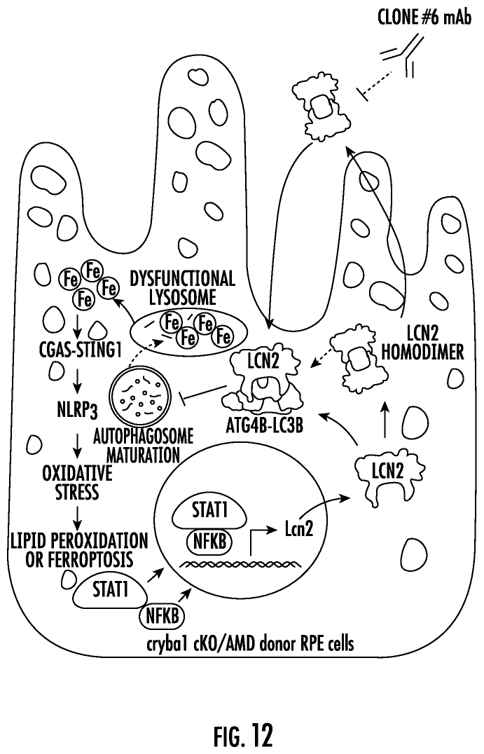

To further show that iron accumulation accompanied by dysregulation of LCN-2 dependent iron chelation in RPE cells could trigger lipid peroxidation, a known trigger for ferroptosis induction (Li et al., Cell Death Dis. 11(2): 88 (2021)), ARPE19 cells were cultured in the presence of iron-rich medium containing FAC and infected with an Ad-LCN-2 construct in the presence of ChQ, followed by live cell imaging with liperfluo dye to assess the extent of lipid peroxidation (Luchi et al., Can. J Physiol. Pharmacol., 97(10): 999-1005 (2019)). The results showed that FAC and Ad-LCN-2 treatment in the presence of ChQ induced lipid peroxidation in RPE cells and also in RAS-selective lethal (RSL3)-treated cells (positive control) (Yang et al., Cell, 156(1-2): 317-331 (2014)), relative to control (FIG. 5J). However, pre-treatment with ferrostatin-1 (Miotto et al., Redox Biol., 28: 101328 (2020)), an inhibitor of lipid peroxidation and ferroptosis followed by treatment with FAC, Ad-LCN-2 and ChQ in the RPE cells could rescue the lipid peroxidation and possibly, ferroptosis induction in these cells (FIG. 5J). Further, to evaluate if targeting LCN-2 can rescue lipid peroxidation activation in vitro, monoclonal antibodies (mAbs) against human recombinant LCN-2 were generated. A specific clone (Clone #6) was chosen from the mAbs pool that recognizes both the monomer and homodimer variants. Treatment with the monoclonal antibody (1 μg/ml), which showed neutralizing activity in the FAC+Ad-LCN-2+ChQ group, could decrease lipid peroxidation in RPE cells, compared to mouse IgG treated cells (FIGS. 5K and 11C). These results suggest that iron accumulation in RPE cells induces oxidative stress and increases lipid peroxidation, which can in turn be rescued by neutralizing LCN-2.

### Example 5

This example demonstrates that the LCN-2 homodimer variant is predominant in an AMD-like mouse model and in dry AMD patients.

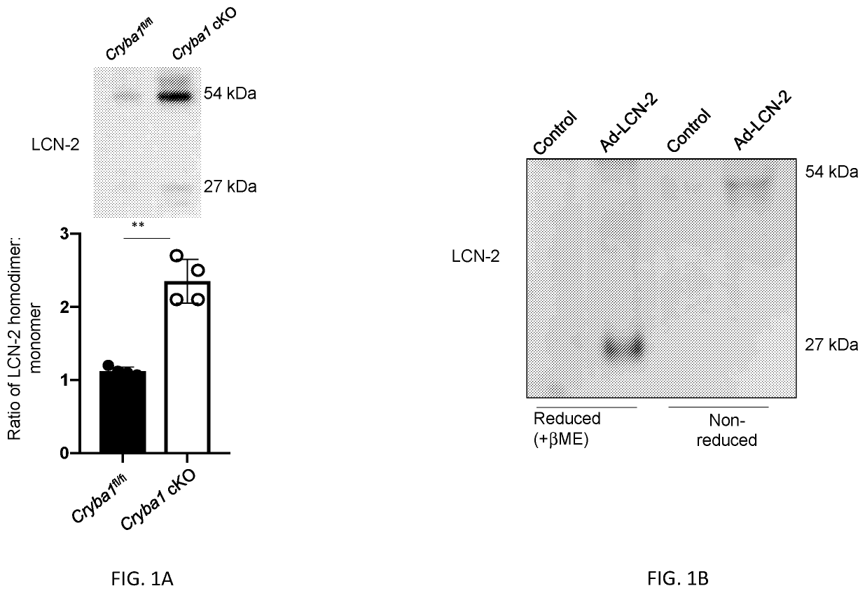

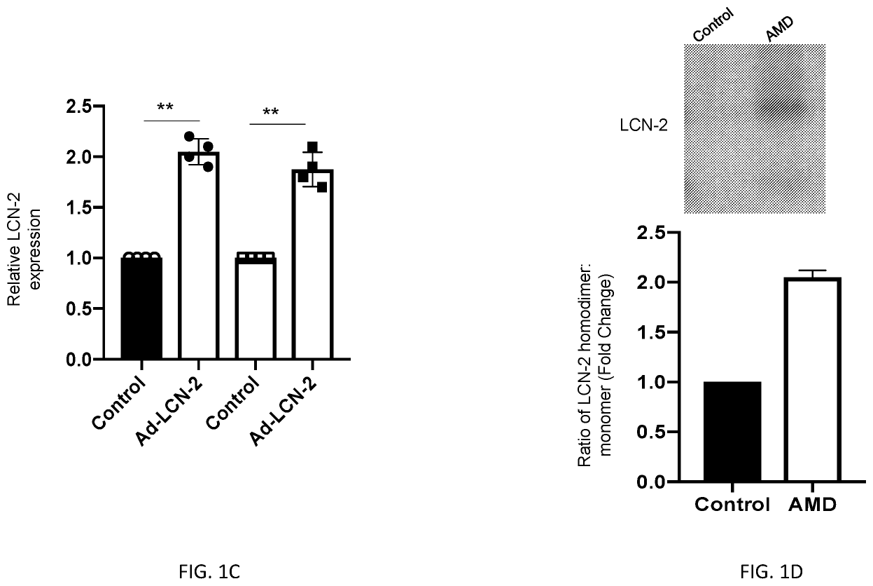

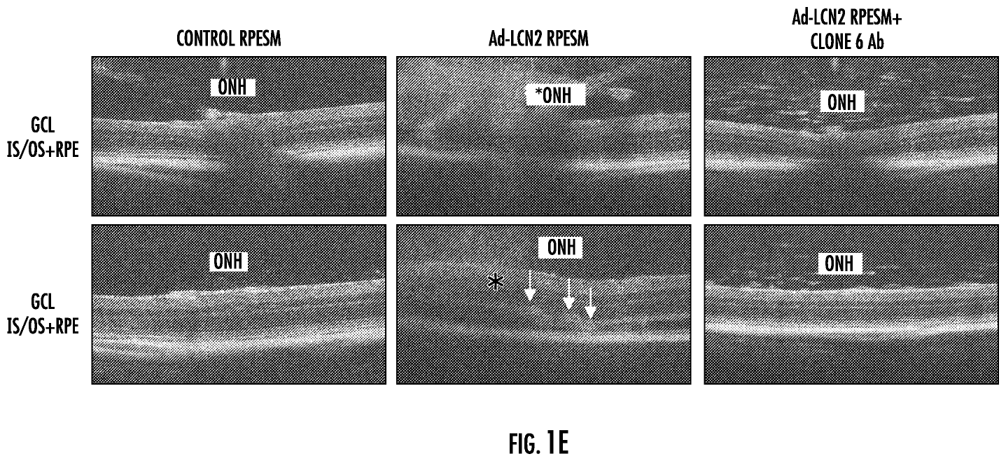

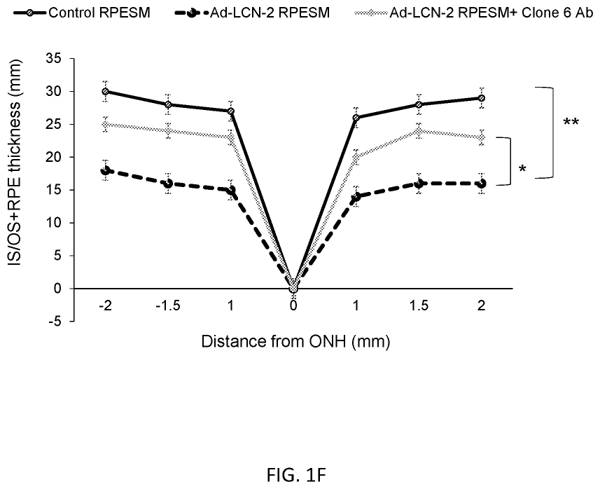

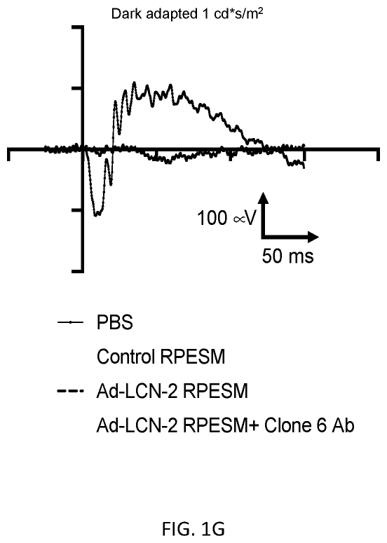

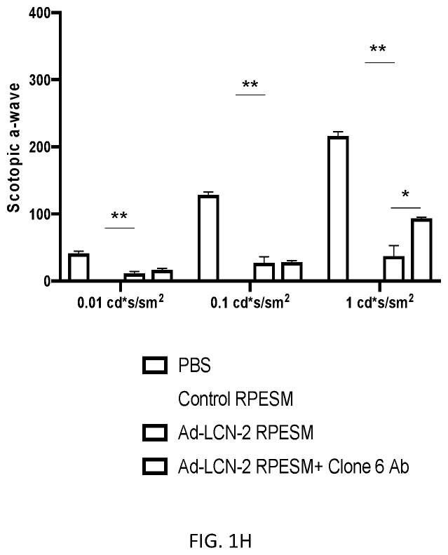

LCN-2 exists in both monomeric and dimeric variants with their relative abundance differing amongst cell types and diseases (Santiago-Sinchez et al., Int. J Mol. Sci., 21(12): 4365 (2020); Barasch et al., Nat. Commun., 7: 12973 (2016)). Previous reports suggest that the LCN-2 homodimer is linked to the pathogenesis of several diseases (Singh et al., Cell Mol. Gastroenterol. Hepatol., 2(4): 482-498.e6 (2016); Rehwald et al., Br. J Cancer, 122(3): 421-433 (2020)). It has been shown that sub-retinal injection of recombinant LCN-2 protein to immune-compromised NOD-SCID mice triggers retinal degeneration (Ghosh et al., Commun. Biol., 2: 348 (2019)). To further investigate whether the LCN-2 homodimer variant plays a pathologic role in dry AMD, Western blot analysis was performed using RPE lysates from aged (12 month-old) Cryba1 cKO and Cryba1-floxed mice with the mAb of Example 4 under both non-reducing and reducing conditions to confirm the presence of the homodimer. It was found that the LCN-2 homodimer variant was increased in the RPE from Cryba1 cKO mice compared to floxed controls (FIG. 1A, FIG. 1J). The monomer was also upregulated in the Cryba1 cKO RPE, but homodimer upregulation was more pronounced relative to controls (FIG. 1A, 1J), and the homodimer to monomer ratio was significantly increased (FIG. 1A, 1J). Further, after overexpressing Adenovirus-LCN-2 (Ad-LCN-2 construct) in floxed RPE explants, only the homodimer variant was secreted into the spent medium (SM) (FIGS. 1B-1C), indicating that the homodimer variant is formed when LCN-2 is upregulated in RPE cells. Increased LCN-2 homodimer was observed in the non-denatured lysates from human AMD donor RPE compared to age-matched controls. The homodimer to monomer ratio was also increased in these lysates (FIGS. 1D and 10A). Taken together, these results indicate that the LCN-2 homodimer variant is significantly upregulated in the RPE of both Cryba1 cKO mice and human AMD patients.

To evaluate further if the LCN-2 homodimer contributes to retinal degeneration, cultured floxed RPE explants were either infected with Ad-LCN-2 construct or left untreated. The RPE SM from the LCN-2 overexpressing explants, which contains only the homodimer variant, was collected and then sub-retinally injected into NOD-SCID mice. Similar mice were injected with RPE SM from untreated explants, or RPE SM from explants infected with Ad-LCN and pre-treated with the mAb of Example 4 (1 μg/ml for 1 h). After one month, using spectral domain optical coherence tomography (SD-OCT) imaging, Ad-LCN-2 SM-treated eyes had altered retinal structure, with changes in inner segment (IS)/outer segment (OS)+RPE layer thickness, when compared to control RPE SM-treated eyes (arrows; FIG. 1E-1F). Ad-LCN-2 RPE SM pretreated with the mAb of Example 4 did not produce alterations in the IS/OS+RPE layer (FIG. 1E-1F). These changes correlated with retinal function, as assessed by electroretinography (ERG), showing decreased scotopic a- and b-wave responses in retinas from mice treated with Ad-LCN-2 SM (FIG. 1G-1I), whereas mAb pretreatment partially rescued such changes (FIG. 1G-1I). Moreover, computer modeling confirmed that the LCN-2 homodimer would not form the complex with ATG4B and LC3 since the dimeric molecule would compete with LC3 for the same binding area at the ATG4B surface. This prompted further investigation into if targeting LCN-2 variants with the mAb of Example 4 in vivo could alleviate the early/dry AMD-like phenotype in a mouse model.

### Example 6

This example demonstrates that the monoclonal antibody (Clone #6) of Example 4 revives autophagy, diminishes inflammasome activation and lipid peroxidation, and restores retinal function in an AMD-like mouse model.

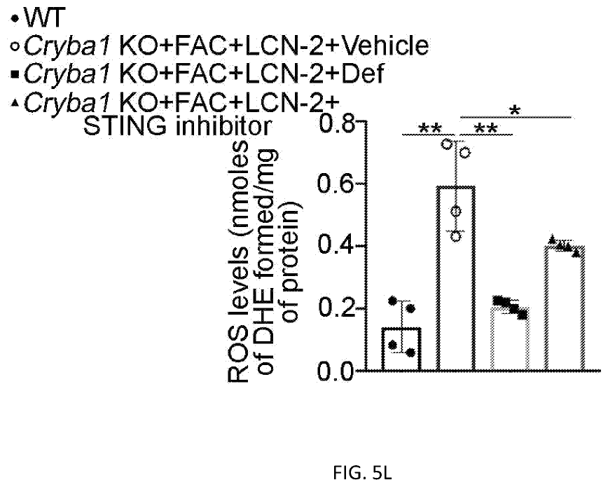

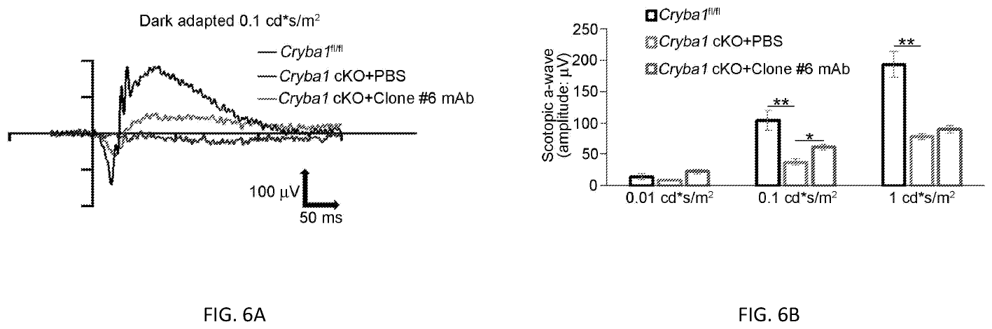

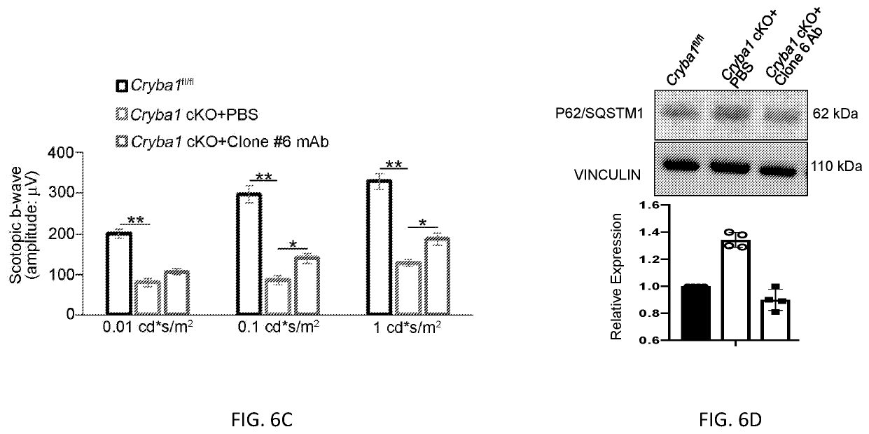

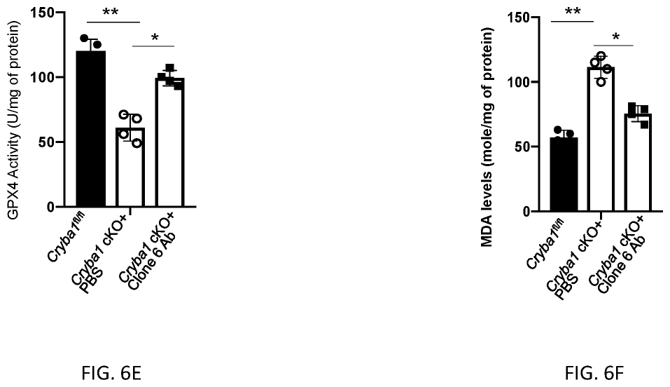

The mAb of Example 4 recognized both LCN-2 variants and shows neutralizing potency along with a noticeable in vitro efficiency in limiting ferroptosis in RPE cells following iron accumulation. One μg/μl of the mAb was subretinally injected into one eye of 5-month-old Cryba1 cKO mice, with the contralateral eye receiving PBS vehicle. Since it was previously showed that retinal function (ERG) in the Cryba1 cKO mouse model is altered from 7 months of age relative to age-matched floxed littermate controls (Valapala et al., Autophagy, 10: 480-496 (2014)), ERGs were performed 2.5 months post-injection. It was found that the LCN-2 antibody improved the scotopic a- and b-waves in the cKO animals (FIGS. 6A-6C and 11D). It was also found that the accumulation of the autophagosome marker (Shang et al., Aging Cell, 16(2): 349-359 (2017)), SQSTM1 (FIGS. 6D and 11B), along with the upregulation of lipid peroxidation marker, MDA (FIG. 6F) and the decrease in the antioxidant enzyme glutathione peroxidase activity (FIGS. 6E and 11A) were rescued to near normal levels in the RPE of Cryba1 cKO eyes that were treated with the mAb as compared to the PBS treated contralateral eyes (FIGS. 6D-6F).

### Example 7

This example demonstrates the generation of the LCN-2 neutralizing monoclonal antibody (Clone #6) of Examples 4-6.

Monoclonal antibodies (mAbs) to human recombinant LCN-2 were generated. The monoclonal anti-LCN-2 was prepared by hybridoma technology. First, RNA was isolated from hybridomas and then the light chain and heavy chain sequences were amplified, followed by PCR and confirmation by Sanger sequencing. The obtained sequences were annotated by using databases. The LCN-2 neutralizing monoclonal antibody (Clone #6) of Examples 4-6 was generated as follows. RNA was isolated from a hybridoma and converted to cDNA. The hypervariable region of the light and heavy chains were amplified using various primers.

The general schematic of the light chain sequence is shown in FIG. 7A. The forward and reverse primer sequences for a product of 350 bp were as follows: V_{L}-Forward: GATATTGTGCTCGACCCAGTCTCCA (SEQ ID NO: 2), V_{L}-Reverse: GGATACAGTTGGTGCAGCATC (SEQ ID NO: 3). The PCR product was analyzed by agarose gel electrophoresis, which showed positive bands at 350 bp molecular weight.

The general schematic of the heavy chain sequence is shown in FIG. 7B. The forward and reverse primer sequences for a product of 440 bp were as follows: V_{H}-Forward: ATGGGATGGAGCTGGATC (SEQ ID NO: 4), V_{H}-Reverse: ATAGACAGATGGGGGTGTCGTTTTGGC (SEQ ID NO: 5). The PCR product was analyzed by agarose gel electrophoresis, which showed positive bands at 440 bp molecular weight.

The amplicons were cloned into pGEM®-T Easy Vector Systems (Promega). Sanger sequencing of multiple clones was carried out. The sequence obtained was annotated in various databases. The amino acid sequences of the various components of monoclonal antibody Clone #6 are set forth in Table 1.

The heavy chain CDR3 and light chain CDR3 amino acid sequences for Clone #6 contain a predicted “gap residue” (denoted in Table 1 as “X”), as predicted using the MiXCR online sequencing tool (described at mixcr.readthedocs.io/en/master/appendix.html). The predicted “gap residue” may be absent or may be any one naturally occurring amino acid residue. The naturally occurring amino acid residues include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

TABLE 1

| | | SEQ |
|---|---|---|
| mAb Clone #6 Anti- | | ID |
| body component | Amino acid sequence | NO |
| heavy chain frame- | QVTLKESGPGILQPSQTLSLTCSFS | 6 |
| work region (FR) 1 | | |
| heavy chain CDR1 | GFSLSSSGMG | 7 |
| heavy chain FR2 | VGWIRQPSGKGLEWLTH | 8 |
| heavy chain CDR2 | IWWDDDK | 9 |
| heavy chain FR3 | RYNPALKSRLTISKDTSNNQVFLKIASVDAAD | 10 |
| | TATYY | |
| heavy chain CDR3 | WSNNIWWXPYYFDYW, wherein X is absent or | 11 |
| | is any naturally occurring amino acid residue. | |
| | In an embodiment, X is proline in SEQ ID NO: 11. | |
| heavy chain FR4 | GQGTTITVSS | 12 |
| light chain FR1 | DIVLTQSPASLAVSLGQRATISYRAS | 13 |
| light chain CDR1 | KSVSTSGYSY | 14 |
| light chain FR2 | MHWNQQKPGQPPRLLIY | 15 |
| light chain CDR2 | LVS | 16 |
| light chain FR3 | NLESGVPARFSGSGSGTDFTLNIHPVEEEDAA | 17 |
| | TYY | |
| light chain CDR3 | CQHIRXAYTF, wherein X is absent or is any | 18 |
| | naturally occurring amino acid residue. In an | |
| | embodiment, X is glutamic acid in SEQ ID NO: 18. | |
| light chain FR4 | GGGTKLEIK | 19 |
| Variable region of | QVTLKESGPGILQPSQTLSLTCSFSGFSLSSSG | 20 |
| heavy chain | MGVGWIRQPSGKGLEWLTHIWWDDDKRYN | |
| | PALKSRLTISKDTSNNQVFLKIASVDAADTAT | |
| | YYWSNNIWWXPYYFDYWGQGTTITVSS, | |
| | wherein X is absent or is any naturally occurring | |
| | amino acid residue. In an embodiment, X is proline | |
| | in SEQ ID NO: 20. | |
| Variable region of | DIVLTQSPASLAVSLGQRATISYRASKSVSTSG | 21 |
| light chain | YSYMHWNQQKPGQPPRLLIYLVSNLESGVPA | |
| | RFSGSGSGTDFTLNIHPVEEEDAATYYCQHIR | |
| | XAYTFGGGTKLEIK, wherein X is absent or is | |
| | any naturally occurring amino acid residue. In an | |
| | embodiment, X is glutamic acid in SEQ ID NO: 21. | |

### Example 8

This example describes a LCN-2 neutralizing monoclonal antibody (Clone #9).

Another specific clone (Clone #9) was chosen from the mAbs pool described in Example 4 that recognizes both the monomer and homodimer variants of LCN-2. Experimental observations similar to those described for mAb Clone #6 were also observed with mAb Clone #9. The amino acid sequences of the various components of monoclonal antibody Clone #9 are set forth in Table 2.

TABLE 2

| mAb Clone #9 Antibody | | SEQ |
|---|---|---|
| component | Amino acid sequence | ID NO: |
| heavy chain framework | QAVVTQESALTTSPGETVTLTCRSS | 22 |
| region (FR) 1 | | |
| heavy chain CDR1 | TGAVTTSNY | 23 |
| heavy chain FR2 | ANWVQEKPDHLFTGLIG | 24 |
| heavy chain CDR2 | GTN | 25 |
| heavy chain FR3 | NRAPGVPARFSGSLIGDKAALTITGAQTEDE | 26 |
| | AIYF | |
| heavy chain CDR3 | CALWYSNHLVF | 27 |
| heavy chain FR4 | GGGTKLTVL | 28 |
| light chain FR1 | QVTLKESGPGILQPSQTLSLTCSFS | 29 |
| light chain CDR1 | GFSLSSSGMG | 30 |
| light chain FR2 | VGWIRQPSGKGLEWLTH | 31 |
| light chain CDR2 | IWWDDDK | 32 |
| light chain FR3 | RYNPALKSRLTISKDTSNNQVFLKIASVDA | 33 |
| | ADTATYY | |
| light chain CDR3 | CGRIIYGGLYYFDYW | 34 |
| light chain FR4 | GQGTTITVSS | 35 |
| Variable region of | QAVVTQESALTTSPGETVTLTCRSSTGAVT | 36 |
| heavy chain | TSNYANWVQEKPDHLFTGLIGGTNNRAPG | |
| | VPARFSGSLIGDKAALTITGAQTEDEAIYFC | |
| | ALWYSNHLVFGGGTKLTVL | |
| Variable region of | QVTLKESGPGILQPSQTLSLTCSFSGFSLSSS | 37 |
| light chain | GMGVGWIRQPSGKGLEWLTHIWWDDDKR | |
| | YNPALKSRLTISKDTSNNQVFLKIASVDAA | |
| | DTATYYCGRIIYGGLYYFDYWGQGTTITVS | |
| | S | |

### Example 9

This example demonstrates that the monoclonal antibody (Clone #9) of Example 8 rescues retinal function and autophagosome accumulation in the Cryba1 cKO mouse model.

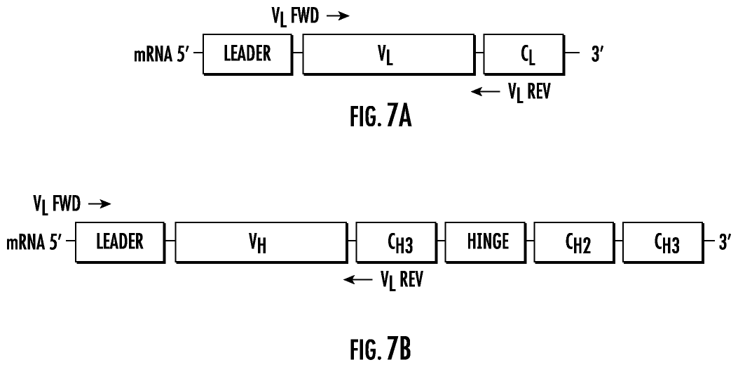

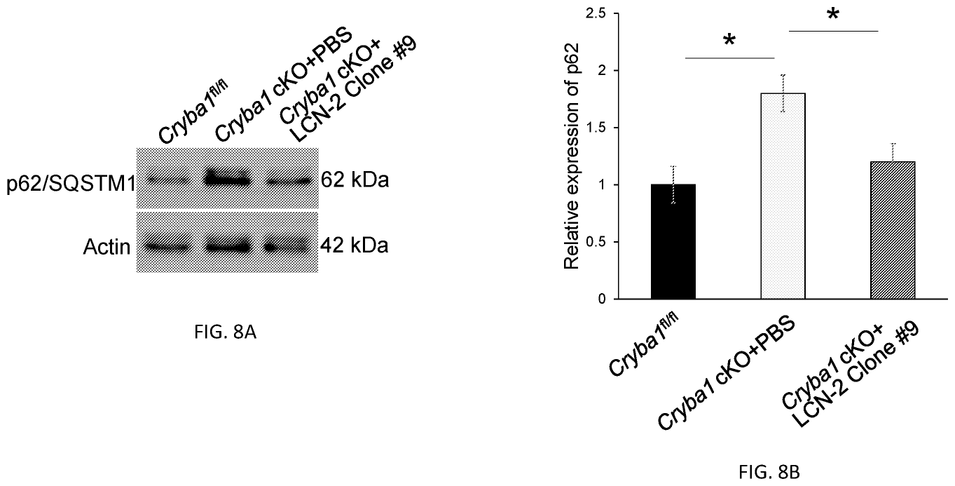

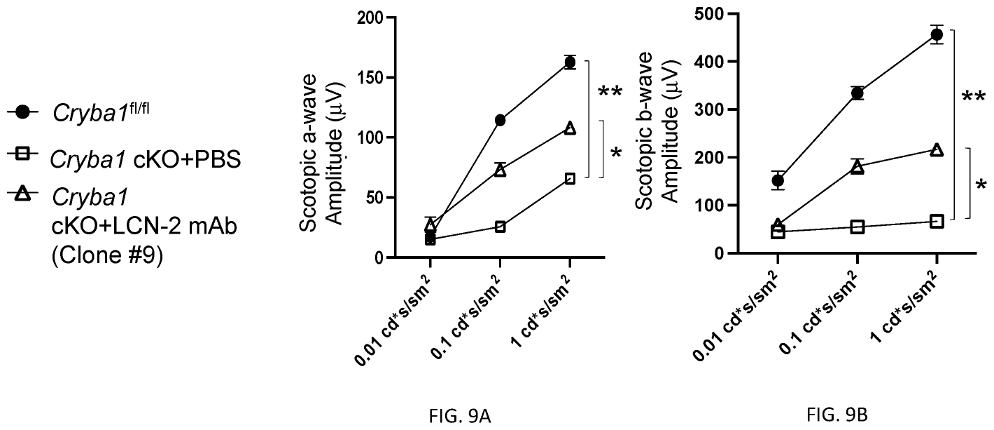

One μg/μl of the mAb (Clone #9) was subretinally injected into one eye of 5-month-old Cryba1 cKO mice, with the contralateral eye receiving PBS vehicle. Since it was previously shown that retinal function (ERG) in the Cryba1 cKO mouse model is altered from 7 months of age relative to age-matched floxed littermate controls (Valapala et al., Autophagy, 10: 480-496 (2014)), ERGs were performed 2.5 months post-injection. It was found that the LCN-2 antibody improved the scotopic a- and b-waves in the cKO animals (FIGS. 9A-9B). It was also found that the accumulation of the autophagosome marker (Shang et al., Aging Cell, 16(2): 349-359 (2017)) SQSTM1 (FIG. 8A) were rescued to near normal levels in the RPE of Cryba1 cKO eyes that were treated with the mAb as compared to the PBS treated contralateral eyes (FIGS. 8A-8B).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified monoclonal antibody, or an antigen binding portion thereof, which neutralizes human Lipocalin 2 (LCN-2).

2. The antibody, or antigen binding portion thereof, of claim 1, wherein the antibody and antigen binding portion thereof neutralize one or both of human LCN-2 monomer and human LCN-2 homodimer.

3. The antibody, or antigen binding portion thereof, of claim 1, wherein human LCN-2 consists of the amino acid sequence of:
(SEQ ID NO: 1)
MPLGLLWLGLALLGALHAQAQDSTSDLIPAPPLSKVPLQQNFQDNQFQGK
WYVVGLAGNAILREDKDPQKMYATIYELKEDKSYNVTSVLFRKKKCDYWI
RTFVPGCQPGEFTLGNIKSYPGLTSYLVRVVSTNYNQHAMVFFKKVSQNR
EYFKITLYGRTKELTSELKENFIRFSKSLGLPENHIVFPVPIDQCIDG.

4. The antibody, or antigen-binding portion of antibody of claim 1, wherein the antigen-binding portion is a Fab fragment (Fab), F(ab′)2 fragment, Fab′ fragment, Fv fragment, scFv, diabody, triabody, tetrabody, or minibody.

5. The antibody, or antigen binding portion thereof, of claim 1 comprising:
(A) the heavy chain complementary determining region (VH CDR) 1 amino acid sequence of SEQ ID NO: 7;
the VH CDR2 amino acid sequence of SEQ ID NO: 9;
the VH CDR3 amino acid sequence of SEQ ID NO: 11;
the light chain complementary determining region (VL CDR) 1 amino acid sequence of SEQ ID NO: 14;
the VL CDR2 amino acid sequence of SEQ ID NO: 16; and
the VL CDR3 amino acid sequence of SEQ ID NO: 18; or
(B) the VH CDR1 amino acid sequence of SEQ ID NO: 23;
the VH CDR2 amino acid sequence of SEQ ID NO: 25;
the VH CDR3 amino acid sequence of SEQ ID NO: 27;
the VL CDR1 amino acid sequence of SEQ ID NO: 30;
the VL CDR2 amino acid sequence of SEQ ID NO: 32; and
the VL CDR3 amino acid sequence of SEQ ID NO: 34.

6. The antibody, or antigen binding portion thereof, of claim 1 comprising:
(A) the heavy chain variable region (VH) amino acid sequence of SEQ ID NO: 20 and the light chain variable region (VL) amino acid sequence of SEQ ID NO: 21; or
(B) the VH amino acid sequence of SEQ ID NO: 36 and the VL amino acid sequence of SEQ ID NO: 37.

7. A nucleic acid comprising a nucleotide sequence encoding the antibody, or antigen binding portion thereof, of claim 1.

8. A recombinant expression vector comprising the nucleic acid of claim 7.

9. A host cell comprising the recombinant expression vector of claim 8.

10. A population of host cells comprising at least two host cells of claim 9.

11. A pharmaceutical composition comprising:
the antibody, or the antigen-binding portion of the antibody, of claim 1,
a nucleic acid comprising a nucleotide sequence encoding the antibody, or antigen binding portion thereof,
a recombinant expression vector comprising the nucleic acid,
a host cell comprising the recombinant expression vector,
or a population of host cells comprising at least two of the host cells,
and a pharmaceutically acceptable carrier.

12. A method of reducing or preventing ferroptotic death of retinal pigmented epithelium (RPE) cells in a subject in need thereof, the method comprising administering:
the antibody, or the antigen-binding portion of the antibody, of claim 1,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject, optionally wherein the antibody, antigen-binding portion of the antibody, nucleic acid, recombinant expression vector, host cell, population of host cells, or pharmaceutical composition is administered by subretinal injection, intravitreal injection, or topical administration.

13. A method of reducing or preventing one or both of lipid peroxidation and inflammasome activation in retinal pigmented epithelium (RPE) cells in a subject in need thereof, the method comprising administering:
the antibody, or the antigen-binding portion of the antibody, of claim 1,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject.

14. A method of reducing or preventing a decrease in one or both of scotopic a- and b-wave responses in a retina of a subject in need thereof, the method comprising administering:
the antibody, or the antigen-binding portion of the antibody, of claim 1,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject, wherein the scotopic a- and b-wave responses in the retina are measured by electroretinography (ERG).

15. A method of increasing one or both of scotopic a- and b-wave responses in a retina of a subject in need thereof, the method comprising administering:
the antibody, or the antigen-binding portion of the antibody, of claim 1,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject, wherein the scotopic a- and b-wave responses in the retina are measured by electroretinography (ERG).

16. A method of increasing one or both of autophagy and glutathione peroxidase activity in RPE cells in a subject in need thereof, the method comprising administering:
the antibody, or the antigen-binding portion of the antibody, of claim 1,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject.

17. The method of claim 12, wherein the subject has age-related macular degeneration (AMD) or is at risk for developing AMD.

18. A method of treating or preventing age-related macular degeneration (AMD) in a subject in need thereof, the method comprising administering:
the antibody, or the antigen-binding portion of the antibody, of claim 1,
a nucleic acid comprising a nucleotide sequence encoding the antibody, or antigen binding portion thereof,
a recombinant expression vector comprising the nucleic acid,
a host cell comprising the recombinant expression vector,
a population of host cells comprising at least two of the host cells, or
a pharmaceutical composition comprising the antibody, or the antigen-binding portion of the antibody, the nucleic acid, the recombinant expression vector, the host cell, or the population of host cells, and a pharmaceutically acceptable carrier,
to the subject.

19. The method of claim 17, wherein the AMD is atrophic AMD, optionally wherein the atrophic AMD includes geographic atrophy.

20. The method of claim 17, wherein the AMD is wet AMD.

21. (canceled)
